(19) United States
(12) Patent Application Publication
(10) Pub. No.: US 2009/0078312 A1
(43) Pub. Date: Mar. 26, 2009

(54) VERFAHREN ZUR HERSTELLUNG VON MIT RYLENTETRACARBONSAEUREDIIMIDEN BESCHICHTETEN SUBSTRATEN

(75) Inventors: Martin Konemann, Mannheim (DE); Torsten Noe, Neustadt (DE); Zhenan Bao, Stanford, CA (US); Joon Hak Oh, Palo Alto, CA (US)

Correspondence Address: OBLON, SPIVAK, MCCLELLAND MAIER & NEUSTADT, P.C., 1940 DUKE STREET, ALEXANDRIA, VA, 22314, US

(73) Assignees: BASF SE, Ludwigshafen (DE); The board of Trustees of the Leland Stan. Jr. Uni., Palo Alto, CA (US)

(21) Appl. No.: 12/212,199

(22) Filed: Sep. 17, 2008

(30) Foreign Application Priority Data
Sep. 18, 2007 (EP) 07116665

(51) Int. Cl. H01L 31/042; C07D 471/04; H01L 51/05; H01J 1/63; H01L 51/40

(52) U.S. Cl. 136/256; 546/26; 546/37; 257/40; 313/504; 438/99; 257/E31.003; 257/E51.005

(57) ABSTRACT

The present invention relates to a process for producing a substrate coated with rylenetetracarboximides, in which a substrate is treated with an N,N′-bisubstituted rylenetetracarboximide and the treated substrate is heated to a temperature at which the N,N′-bisubstituted rylenetetracarboximide is converted to the corresponding N,N′-unsubstituted compound. The present invention further relates to semiconductor units, organic solar cells, excitonic solar cells and organic light-emitting diodes which comprise a substrate produced by this process. The present invention further relates to a process for preparing N,N′-unsubstituted rylenetetracarboximides, in which the corresponding N,N′-bisubstituted rylenetetracarboximides are provided and heated to a temperature at which these compounds are converted to the corresponding N,N′-unsubstituted compounds.

The present invention relates to a process for producing a substrate coated with rylenetetracarboximides, in which a substrate is treated with an N,N′-bisubstituted rylenetetracarboximide and the treated substrate is heated to a temperature at which the N,N′-bisubstituted rylenetetracarboximide is converted to the corresponding N,N′-unsubstituted compound. The present invention further relates to semiconductor units, organic solar cells, excitonic solar cells and organic light-emitting diodes which comprise a substrate produced by this process. The present invention further relates to a process for preparing N,N′-unsubstituted rylenetetracarboximides, in which the corresponding N,N′-bisubstituted rylenetetracarboximides are provided and heated to a temperature at which these compounds are converted to the corresponding N,N′-unsubstituted compounds.

For the future it is expected that not only the conventional inorganic semiconductors but increasingly also organic semiconductors based on low molecular weight or polymeric materials will be used in many sectors of the electronics industry. In many cases, these organic semiconductors have advantages over the conventional inorganic semiconductors, for example better substrate compatibility and better processibility of the semiconductor components based on them. They allow processing on flexible substrates and enable their interface orbital energies to be adjusted precisely to the particular application sector by the methods of molecular modeling. The significantly reduced costs of such components have brought a renaissance to the field of organic electronics. “Organic electronics” is concerned principally with the development of novel materials and manufacturing processes for the production of electronic components based on organic semiconductor layers. These include in particular organic field-effect transistors (OFETs) and organic light-emitting diodes (OLEDs; for example for use in displays) and organic photovoltaics. Great potential for development is also ascribed to organic field-effect transistors, for example in memory elements and integrated optoelectronic devices. There is therefore a great need for organic compounds which are suitable as organic semiconductors, especially n-type semiconductors, and specifically for use in organic field-effect transistors and solar cells.

The direct conversion of solar energy to electrical energy in solar cells is based on the internal photoeffect of a semiconductor material, i.e. the generation of electron-hole pairs by absorption of photons and the separation of the negative and positive charge carriers at a p-n transition or a Schottky contact. The photovoltage thus generated, in an external circuit, can bring about a photocurrent through which the solar cell releases its power.

The semiconductor can absorb only those photons which have an energy which is greater than its band gap. The size of the semiconductor band gap thus determines the proportion of sunlight which can be converted to electrical energy. It is expected that, in the future, organic solar cells will outperform the conventional solar cells based on silicon owing to lower costs, a lighter weight, the possibility of producing flexible and/or colored cells, the greater possibility of fine adjustment of the band gap. There is thus a great need for organic semiconductors which are suitable for producing organic solar cells.

Solar cells normally consist of two absorbent materials with different band gaps, in order to utilize the solar energy with maximum efficiency. The first organic solar cells consisted of a two-layer system composed of a copper phthalocyanine as the p-conductor and PTCBI as the n-conductor, and exhibited an efficiency of 1%. In order to utilize as many incident photons as possible, relatively high layer thicknesses are used (e.g. 100 nm). In order to generate electricity, the excited state generated by the absorbed photons must, however, reach a p-n junction, in order to generate a hole and an electron, which then flow to the anode and cathode. However, most organic semiconductors only have diffusion lengths for the excited state of up to 10 nm. Even by virtue of the best production processes known to date, the distance over which the excited state has to be transmitted cannot be reduced to values of below 10 to 30 nm.

WO 2007/093643 describes, inter alia, N,N′-unsubstituted, fluorinated rylenetetracarboximides, a process for preparation thereof and the use thereof, especially as n-type semiconductors.

EP 07110133.1 (=PCT/EP 2008/053063), which was unpublished at the priority date of the present application, describes the advantageous properties of N,N′-unsubstituted rylenetetracarboximides for use in organic electronics.

PCT/EP/2007/058303 (=WO 2008/017714), which was unpublished at the priority date of the present application, (and Adv. Mat. 2007, 19, 1123-1127) describe N,N′-unsubstituted perylenetetracarboximides as good n-semiconductors in organic field-effect transistors (OFETs). These compounds are air-stable, and have a good field-effect mobility. A process which allows the particularly suitable N,N′-unsubstituted perylenetetracarboximides to be processed from solution is, however, not demonstrated.

Chem. Mater. 2006, 18, 3715-3726 and PCT/EP2007/053330 (=WO 2007/116001), which was unpublished at the priority date of the present application, describe N,N′-substituted rylenetetracarboximide compounds which are processible in liquid form. The defined thermal elimination of the substituents of the imido groups is not described herein.

The charge mobilities of the N,N′-substituted rylenetetracarboximide compounds known from the prior art are, however, in need of improvement. In contrast, N,N′-unsubstituted rylenetetracarboximide compounds frequently have high charge mobilities, but are sparingly soluble or completely insoluble in solvents, which does not permit wet processing directly.

It was therefore an object of the present invention to provide a process for producing substrates coated at least partly with N,N′-unsubstituted rylenetetracarboximide compounds, which can be performed simply and inexpensively.

This object is achieved by a process for producing a substrate coated at least partly with a compound of the formula (I)

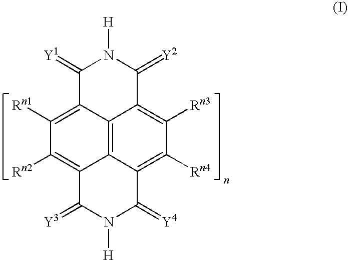

in which
n is an integer from 1 to 8
Y¹, Y², Y³ and Y⁴ are each independently O or S and
Rⁿ¹, Rⁿ², Rⁿ³ and Rⁿ⁴ are each independently hydrogen, F, Cl, Br, CN, alkoxy, alkylthio, alkylamino, dialkylamino, aryloxy, arylthio, hetaryloxy or hetarylthio, where
   two of the Rⁿ¹ and Rⁿ² radicals and/or Rⁿ³ and Rⁿ⁴ radicals in each case may together also be part of an aromatic ring system fused to one or two adjacent naphthalene units of the rylene skeleton;
      in which
i) at least one compound of the formula (II) is provided

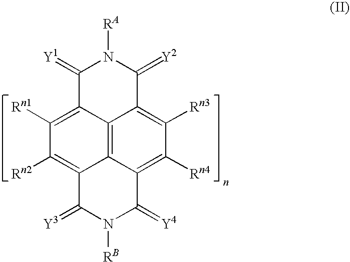

in which
n, Y¹, Y², Y³, Y⁴, Rⁿ¹, Rⁿ², Rⁿ³ and Rⁿ⁴ are each as defined for the compound of the formula (I) and
R^{A} and R^{B} are each independently a group of the formula (III)

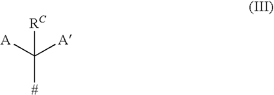

in which
# in each case represents the bond to the nitrogen atom,
A and A′ are each independently unsubstituted or substituted C₁-C₂₅-alkyl, unsubstituted or substituted C₂-C₂₅-alkenyl, unsubstituted or substituted C₂-C₂₅-alkynyl, unsubstituted or substituted aryl or unsubstituted or substituted hetaryl, where C₁-C₂₅-alkyl, C₂-C₂₅-alkenyl and C₂-C₂₅-alkynyl may each be interrupted once or more than once by O, S, NR^{a}, —C(═O)—, —C(═O)O—, —C(═O)N(R^{a})—, —S(═O)₂O— or —S(═O)₂N(R^{a})—, in which
   R^{a} is selected from unsubstituted or substituted C₁-C₁₂-alkyl, unsubstituted or substituted aryl and unsubstituted or substituted hetaryl, and
   R^{c} is hydrogen or unsubstituted or substituted C₁-C₁₂-alkyl, unsubstituted or substituted C₂-C₁₂-alkenyl, unsubstituted or substituted C₂-C₁₂-alkynyl, unsubstituted or substituted aryl or unsubstituted or substituted hetaryl, where C₁-C₂₅-alkyl, C₂-C₂₅-alkenyl and C₂-C₂₅-alkynyl may each be interrupted once or more than once by O, S, NR^{a}, —C(═O)—, —C(═O)O—, —C(═O)N(R^{a})—, —S(═O)₂O— or —S(═O)₂N(R^{a})—,
ii) the substrate is treated with a solution of the compounds of the formula (II) provided and
iii) the treated substrate is heated to a temperature at which at least some of the compounds of the formula (II) are converted to compounds of the formula (I).

By virtue of the use of N,N′-substituted rylenetetracarboximide compounds, the process according to the invention combines the advantages of the processing of rylene compounds in dissolved form with the advantages of gas phase processing. The former include the relatively easy purification of the starting compounds, relatively low material losses in the course of processing and relatively inexpensive processibility. The latter include the provision of compounds with pigmentary character, crystalline order and improved control of the morphology of the layers producible through control of the crystallization temperature.

The present invention further relates to coated substrates obtainable by the above-described process according to the invention.

The present invention further relates to semiconductor units, organic solar cells, excitonic solar cells and organic light-emitting diodes which comprise at least one inventive coated substrate.

The present invention further relates to a process for preparing compounds of the formula (I), as defined above and below, in which
A) a compound of the formula (II) as defined above and below is provided,
B) the compound of the formula (II) is heated to a temperature at which at least some of the compound of the formula (II) is converted to a compound of the formula (I), i.e. at which at least some of the R^{A} and R^{B} groups of the compounds of the formula (II) are exchanged for hydrogen.

The present invention further relates to compounds of the formula (I) and (II) which have not been described to date and which can be used in an advantageous manner in the processes according to the invention and in the inventive coated substrates.

In the compounds of the formulae (I) and (II), n denotes the number of naphthalene units bonded in the peri position, which form the base skeleton of the inventive rylene compounds. In the individual Rⁿ¹ to Rⁿ⁴ radicals, n denotes the particular naphthalene group of the rylene skeleton to which the radicals are bonded. Rⁿ¹ to Rⁿ⁴ radicals which are bonded to different naphthalene groups may each have identical or different definitions. Accordingly, the compounds of the general formulae (I) and (II) may be naphthalenediimides, perylenediimides, terrylenediimides, quaterrylenediimides, pentarylenediimides, hexarylenediimides, heptarylenediimides or octarylenediimides of the following formula:

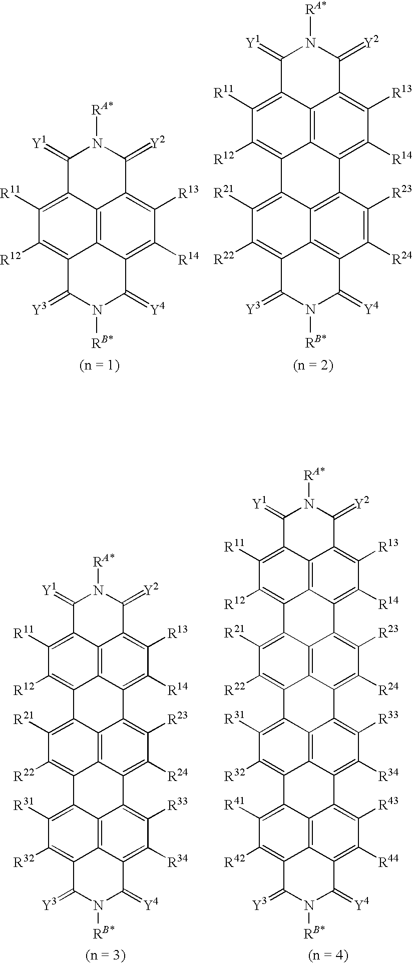

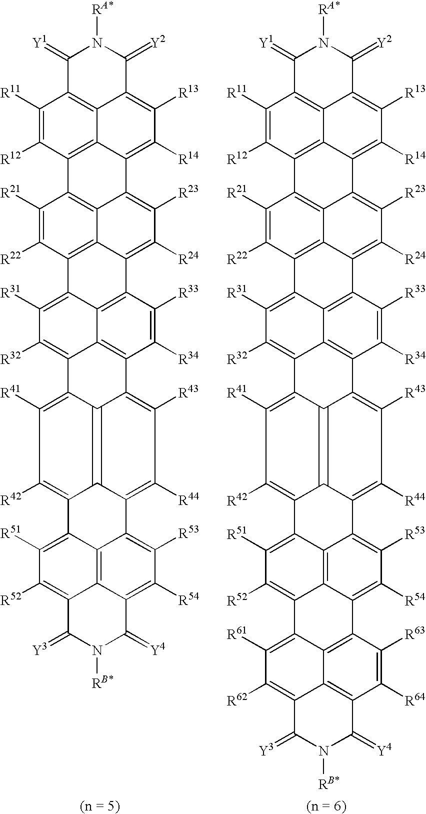

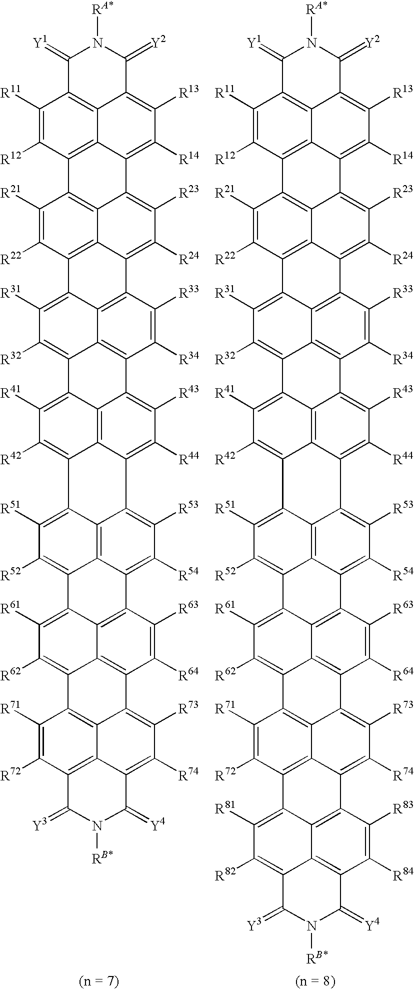

in which R^{A}* and R^{B}* are each hydrogen in the compounds of the formula (I) and each have one of the definitions given for R^{A} and R^{B} in the compounds of the formula (II).

When in each case two of the Rⁿ¹ and Rⁿ² radicals and/or Rⁿ³ and Rⁿ⁴ radicals in the compounds of the formulae (I) and (II) together represent part of an aromatic ring system fused to one or two adjacent naphthalene units of the rylene skeleton, in the case that the two Rⁿ¹ and Rⁿ² radicals and/or Rⁿ³ and Rⁿ⁴ radicals are bonded to the same naphthalene unit they are a group of the formula (IV)

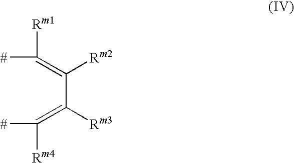

and in the case that the two Rⁿ¹ and Rⁿ² radicals and/or Rⁿ³ and Rⁿ⁴ radicals are bonded to two adjacent naphthalene units they are a group of the formula (IV.a) or (IV.b)

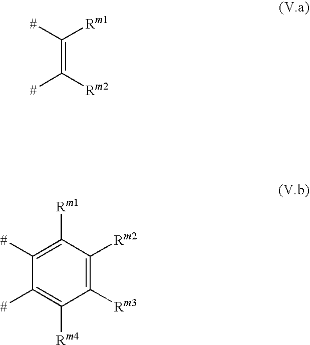

in which # in each case represents a bond to the naphthalene unit of the rylene skeleton. This means that R¹¹ with R¹², R¹³ with R¹⁴; R²¹ with R²²; R²³ with R²⁴, R³¹ with R³², R³³ with R³⁴, R⁴¹ with R⁴², R⁴³ with R⁴⁴, R⁵¹ with R⁵², R⁵³ with R⁵⁴, R⁶¹ with R⁶², R⁶³ with R⁶⁴, R⁷¹ with R⁷², R⁷³ with R⁷⁴, R⁸¹ with R⁸², and R⁸³ with R⁸⁴ may each together be a group of the formula (IV), and R²¹ with R¹², R²³ with R¹⁴, R³¹ with R²², R³³ with R²⁴, R⁴¹ with R³², R⁴³ with R³⁴, R⁵¹ with R⁴², R⁵³ with R⁴⁴, R⁶¹ with R⁵², R⁶³ with R⁵⁴, R⁷¹ with R⁶², R⁷³ with R⁶⁴, R⁸¹ with R⁷², and R⁸³ may each together be a group of the formula (V.a) or (V.b).

In the groups of the formulae (IV), (V.a) and (V.b), the R^{m1}, R^{m2}, R^{m3} and R^{m4} radicals are each independently as defined for Rⁿ¹, Rⁿ², Rⁿ³ and Rⁿ⁴ radicals. The R^{m1}, R^{m2}, R^{m3} and R^{m4} radicals are preferably each hydrogen.

Typically, in the compounds of the formulae (I) and (II), from 0 to 4 times two of the Rⁿ¹ and Rⁿ² radicals and/or Rⁿ³ and Rⁿ⁴ radicals in each case will together be part of a fused, aromatic ring system. Preferably, in the compounds of the formulae (I) and (II), 0, 2 or 4 times two of the Rⁿ¹ and Rⁿ² radicals and/or Rⁿ³ and Rⁿ⁴ radicals in each case will together be part of a fused, aromatic ring system. More preferably, in the case that multiple pairs of Rⁿ¹ and Rⁿ² radicals and/or Rⁿ³ and Rⁿ⁴ radicals together are part of a fused, aromatic ring system, they are selected from exclusively Rⁿ¹ and Rⁿ² or exclusively Rⁿ³ and Rⁿ⁴, i.e. the extension of the rylene ring system is brought about by extending one naphthalene unit in each case or by bridging two naphthalene units in each case.

In the context of the present invention, the expression “alkyl” comprises straight-chain or branched saturated hydrocarbon groups bonded via a carbon atom. It is preferably straight-chain or branched C₁-C₂₅-alkyl and especially C₁-C₁₂-alkyl. Examples of alkyl groups are especially methyl, ethyl, n-propyl, isopropyl, n-butyl, isobutyl, sec-butyl, tert-butyl, n-pentyl, n-hexyl, n-heptyl, n-octyl, n-nonyl, n-decyl, n-undecyl, n-dodecyl, n-tridecyl, n-tetradecyl, n-hexadecyl, n-octadecyl and n-eicosyl.

The expression “alkyl” also comprises alkyl radicals whose carbon chain may be interrupted by one or more nonadjacent groups selected from O, S, NR^{a}, —C(═O)—, —C(═O)O—, —C(═O)N(R^{a})—, —S(═O)₂O— or —S(═O)₂N(R^{a})—, where R^{a} is selected from in each case optionally substituted C₁-C₁₂-alkyl, aryl and hetaryl.

The expression “optionally substituted alkyl” comprises alkyl radicals in which 1 or more and especially from 1 to 6 of the hydrogen atoms of the carbon chain may be replaced by a substituent other than hydrogen. Suitable substituents are, for example, fluorine, chlorine, bromine, CN, NO₂, aryl, hetaryl, OH and SH.

The above remarks regarding alkyl apply correspondingly to the alkyl moieties in alkoxy, alkylthio, alkylamino and dialkylamino.

In the context of the present invention, the expression “aryl” comprises mono- or polycyclic aromatic hydrocarbon radicals which may be unsubstituted or substituted. The expression “aryl” preferably represents phenyl, naphthyl, fluorenyl, anthracenyl or phenanthrenyl, more preferably phenyl or naphthyl and most preferably phenyl, where aryl in the case of substitution may bear generally 1, 2, 3, 4 or 5 and preferably 1, 2 or 3 substituents. Suitable substituents are preferably selected from F, Cl, Br, CN, NO₂, OH, SH, NH₂, COOH, C₁-C₃₀-alkyl, especially C₁-C₁₈-alkyl, C₁-C₁₂-alkoxy, C₁-C₁₂-alkylthio, C₁-C₁₂-alkylamino, C₁-C₁₂-dialkylamino, C₂-C₁₂-alkenyl, C₂-C₁₂-alkynyl, C₁-C₁₂-alkylcarbonyl, C₁-C₁₂-alkoxycarbonyl, C₁-C₁₂-alkylthiocarbonyl, C₁-C₁₂-alkylcarbonyloxy and aryl, where aryl is unsubstituted or mono-, di- or tri-C₁-C₆-alkyl-substituted.

The above remarks regarding aryl apply correspondingly to the aryl moieties in aryloxy and arylthio.

In the context of the present invention, the expression “heteroaryl” comprises unsubstituted or substituted, heteroaromatic, mono- or polycyclic groups, preferably the pyridyl, quinolinyl, acridinyl, pyridazinyl, pyrimidinyl, pyrazinyl, pyrrolyl, imidazolyl, pyrazolyl, indolyl, purinyl, indazolyl, benzotriazolyl, 1,2,3-triazolyl, 1,3,4-triazolyl and carbazolyl groups, where these heterocycloaromatic groups in the case of substitution may bear generally 1, 2 or 3 substituents. Suitable substituents are preferably selected from F, Cl, Br, CN, NO₂, OH, SH, NH₂, COOH, C₁-C₃₀-alkyl, especially C₁-C₁₈-alkyl, C₁-C₁₂-alkoxy, C₁-C₁₂-alkylthio, C₁-C₁₂-alkylamino, C₁-C₁₂-dialkylamino, C₂-C₁₂-alkenyl, C₂-C₁₂-alkynyl, C₁-C₁₂-alkylcarbonyl, C₁-C₁₂-alkoxycarbonyl, C₁-C₁₂-alkylthiocarbonyl, C₁-C₁₂-alkylcarbonyloxy and aryl, where aryl is unsubstituted or mono-, di- or tri-C₁-C₆-alkyl-substituted.

The above remarks regarding heteroaryl apply correspondingly to the heteroaryl moieties in heteroaryloxy and heteroarylthio.

In the context of the present invention, the expression “alkenyl” comprises straight-chain or branched hydrocarbon groups which are bonded via a carbon atom and comprise at least one carbon-carbon double bond. They are preferably straight-chain or branched C₂-C₂₅-alkenyl and especially C₂-C₁₂-alkenyl. Examples of alkenyl groups are especially ethenyl, n-propenyl, isopropenyl, n-butenyl, isobutenyl, sec-butenyl, n-pentenyl, n-hexenyl, n-heptenyl, n-octenyl, n-nonenyl, n-decenyl, n-undecenyl and n-dodecenyl.

The expression “alkenyl” also comprises alkenyl groups whose carbon chain may be interrupted by one or more nonadjacent groups which are selected from O, S, NR^{a}, —C(═O)—, —C(═O)O—, —C(═O)N(R^{a})—, —S(═O)₂O— or —S(═O)₂N(R^{a})—, where R^{a} is selected from in each case optionally substituted C₁-C₁₂-alkyl, aryl and hetaryl.

The expression “optionally substituted alkenyl” comprises alkenyl radicals in which 1 or more and especially from 1 to 6 of the hydrogen atoms of the carbon chain may be replaced by a substituent other than hydrogen. Suitable substituents are, for example, fluorine, chlorine, bromine, CN, NO₂, aryl, hetaryl, OH and SH.

In the context of the present invention, the expression “alkynyl” comprises straight-chain or branched hydrocarbon groups which are bonded via a carbon atom and comprise at least one carbon-carbon triple bond. They are preferably straight-chain or branched C₂-C₂₅-alkynyl and especially C₂-C₁₂-alkynyl. Examples of alkenyl groups are especially ethynyl, n-propynyl, n-butynyl, n-pentynyl, n-hexynyl, n-heptynyl, n-octynyl, n-nonynyl, n-decynyl, n-undecynyl and n-dodecynyl.

The expression “alkynyl” also comprises alkynyl groups whose carbon chain may be interrupted by one or more nonadjacent groups which are selected from O, S, NR^{a}, —C(═O)—, —C(═O)O—, —C(═O)N(R^{a})—, —S(═O)₂O— or —S(═O)₂N(R^{a})—, where R^{a} is selected from in each case optionally substituted C₁-C₁₂-alkyl, aryl and hetaryl.

The expression “optionally substituted alkynyl” further comprises alkynyl radicals in which 1 or more and especially from 1 to 6 of the hydrogen atoms of the carbon chain may be replaced by a substituent other than hydrogen. Suitable substituents are, for example, fluorine, chlorine, bromine, CN, NO₂, aryl, hetaryl, OH and SH.

With regard to the processes according to the invention, preference is given to compounds of the formulae (I) and (II) in which one or more of the n, Y¹, Y², Y³, Y⁴, Rⁿ¹, Rⁿ², Rⁿ³ and Rⁿ⁴ radicals, and for the compounds of the formula (II) also the R^{A} and R^{B} radicals, each independently have one of the definitions given below. For the processes according to the invention, particular preference is given to compounds of the formulae (I) and (II) in which the aforementioned radicals all have one of the definitions given below.

In the compounds of the formula (II), the R^{A} and R^{B} groups may have identical or different definitions. The R^{A} and R^{B} groups in a compound of the formula (II) preferably have the same definition.

The Y¹, Y², Y³ and Y⁴ radicals in the compounds of the formula (I) and (II) are preferably each O.

The Rⁿ¹, Rⁿ², Rⁿ³ and Rⁿ⁴ radicals in the compounds of the formulae (I) and (II) are preferably each independently hydrogen, F, Cl, Br, CN, aryloxy or arylthio. More preferably, from 0 to (2n−2) of the Rⁿ¹, Rⁿ², Rⁿ³ and Rⁿ⁴ radicals in the compounds of the formulae (I) and (II) are each F, Cl, Br, CN, aryloxy or arylthio, and the remaining Rⁿ¹, Rⁿ², Rⁿ³ and Rⁿ⁴ radicals are each hydrogen. In a specific embodiment, all Rⁿ¹, Rⁿ², Rⁿ³ and Rⁿ⁴ radicals in the compounds of the formulae (I) and (II) are hydrogen.

Specific examples of the Rⁿ¹, Rⁿ², Rⁿ³ and Rⁿ⁴ radicals specified in the compounds of the formulae (I) and (II) are as follows:

hydrogen, fluorine, chlorine, bromine and cyano; but also
methoxy, ethoxy, propoxy, isopropoxy, butoxy, isobutoxy, sec-butoxy, tert-butoxy, pentoxy, isopentoxy, neopentoxy, tert-pentoxy and hexoxy;
methylthio, ethylthio, propylthio, isopropylthio, butylthio, isobutylthio, sec-butylthio, tert-butylthio, pentylthio, isopentylthio, neopentylthio, tert-pentylthio and hexylthio;
methylamino, ethylamino, propylamino, isopropylamino, butylamino, isobutylamino, pentylamino, hexylamino, dimethylamino, methylethylamino, diethylamino, dipropyl-amino, diisopropylamino, dibutylamino, diisobutylamino, dipentylamino, dihexylamino, dicyclopentylamino, dicyclohexylamino, dicycloheptylamino, diphenylamino and dibenzylamino;
phenoxy, phenylthio, 2-naphthoxy, 2-naphthylthio, 2-, 3- and 4-pyridyloxy, 2-, 3- and 4-pyridylthio, 2-, 4- and 5-pyrimidyloxy and 2-, 4- and 5-pyrimidylthio.

The R^{A} and R^{B} radicals in the compounds of the formula (I) may have the same definition or different definitions. The R^{A} and R^{B} radicals in the compounds of the formula (I) preferably have the same definition.

The A and A′ radicals in the groups of the formula (III) may have the same definition or different definitions. In a first embodiment, the A and A′ radicals in the groups of the formula (III) have the same definition.

In the groups of the formula (III), at least one of the A or A′ radicals is unsubstituted or substituted C₁-C₂₅-alkyl, unsubstituted or substituted C₃-C₂₅-alkenyl or unsubstituted or substituted C₃-C₂₅-alkynyl, where the three radicals mentioned have at least one hydrogen atom in the beta position to the nitrogen atom of the rylene skeleton, and where C₁-C₂₅-alkyl, C₃-C₂₅-alkenyl and C₃-C₂₅-alkynyl may each be interrupted once or more than once, for example once, twice, thrice or more than thrice, by O, S, NR^{a}, —C(═O)—, —C(═O)O—, —C(═O)N(R^{a})—, —S(═O)₂O— or —S(═O)₂N(R^{a})—, and R^{a} is as defined above.

In addition, in the compounds of the formula (II), at least one of the A or A′ radicals and especially both the A and A′ radicals in the groups of the formula (III) are preferably each independently C₁-C₂₅-alkyl, C₂-C₂₅-alkenyl or C₂-C₂₅-alkynyl, where the aforementioned radicals may each be interrupted once or more than once, for example once, twice, three times, four times or more than four times, by O or S. Preferably at least one of the A and A′ radicals has a hydrogen atom in the beta position to the nitrogen atom of the rylene skeleton.

In a specific embodiment of the present invention, in the compounds of the formula (II), R^{A} and R^{B} are each independently a group of the formula (III.1),

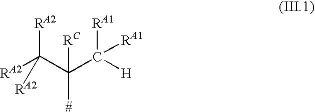

in which
R^{A1} is in each case independently unsubstituted or substituted C₁-C₁₂-alkyl, unsubstituted or substituted C₂-C₁₂-alkenyl, unsubstituted or substituted C₂-C₁₂-alkynyl, unsubstituted or substituted aryl or unsubstituted or substituted hetaryl, where C₁-C₁₂-alkyl, C₂-C₁₂-alkenyl and C₂-C₁₂-alkynyl may each be interrupted once or more than once, for example once, twice, thrice or more than thrice, by O, S, NR^{a}, —C(═O)—, —C(═O)O—, —C(═O)N(R^{a})—, —S(═O)₂O— or —S(═O)₂N(R^{a})—, in which
   R^{a} is selected from unsubstituted or substituted C₁-C₁₂-alkyl, unsubstituted or substituted aryl and unsubstituted or substituted hetaryl,
R^{A2} is in each case independently hydrogen or is as defined for R^{A1};
R^{C} is hydrogen or unsubstituted or substituted C₁-C₁₂-alkyl, unsubstituted or substituted C₂-C₁₂-alkenyl, unsubstituted or substituted C₂-C₁₂-alkynyl, unsubstituted or substituted aryl or unsubstituted or substituted hetaryl, where C₁-C₂₅-alkyl, C₂-C₂₅-alkenyl and C₂-C₂₅-alkynyl may each be interrupted once or more than once, for example once, twice, thrice or more than thrice, by O, S, NR^{a}, —C(═O)—, —C(═O)O—, —C(═O)N(R^{a})—, —S(═O)₂O— or —S(═O)₂N(R^{a})—, and R^{a} is as defined above; and
# in each case represents the bond to the nitrogen atom.

More preferably, A and A′ in the groups of the formula (III) are each independently a —CH(R^{D})(R^{E}) group in which R^{D} and R^{E} are each independently hydrogen or C₁-C₁₂-alkyl, preferably C₁-C₁₂-alkyl and more preferably C₁-C₆-alkyl.

Likewise more preferably, at least one of the A and A′ radicals in the groups of the formula (III) is a —CH(R^{D})(R^{E}) group in which R^{D} and R^{E} are each independently C₁-C₁₂-alkyl, unsubstituted or substituted aryl or unsubstituted or substituted hetaryl, where C₁-C₁₂-alkyl may in each case be interrupted once or more than once, for example once, twice, thrice or more than thrice, by O or S.

In a specific embodiment, at least one of the A and A′ radicals in the groups of the formula (III) is a —CH(R^{D})(R^{E}) group in which R^{D} and R^{E} are each independently C₁-C₁₂-alkyl, unsubstituted or substituted aryl or unsubstituted or substituted hetaryl, and where C₁-C₁₂-alkyl may in each case be interrupted once or more than once, for example once, twice, thrice or more than thrice, by O or S and R^{c} is hydrogen, unsubstituted or substituted C₁-C₁₂-alkyl, unsubstituted or substituted aryl or unsubstituted or substituted hetaryl, where C₁-C₁₂-alkyl may in each case be interrupted once or more than once, for example once, twice, thrice or more than thrice, by or S.

Equally more preferably, in the group of the formula (III), one of the A and A′ radicals is a —CH(R^{D})(R^{E}) group in which R^{D} and R^{E} are each independently C₁-C₁₂-alkyl, unsubstituted or substituted aryl or unsubstituted or substituted hetaryl, and where C₁-C₁₂-alkyl may in each case be interrupted once or more than once by O or S, and the other of the A and A′ radicals is unsubstituted or substituted aryl or unsubstituted or substituted hetaryl. In the case of substitution, aryl and hetaryl bear generally 1, 2 or 3 substituents which are preferably selected from F, Cl, Br, CN, NO₂, OH, SH, NH₂, C₁-C₁₂-alkylamino, C₁-C₁₂-dialkylamino, COOH, C₁-C₁₈-alkyl, C₁-C₁₂-alkoxy, C₁-C₁₂-alkylthio, C₁-C₁₂-alkylcarbonyl, C₁-C₁₂-alkoxycarbonyl, unsubstituted aryl and mono-, di- or tri-C₁-C₆-alkyl substituted alkyl. Especially preferably, in this embodiment, R^{c} is hydrogen, unsubstituted or substituted C₁-C₁₂-alkyl, unsubstituted or substituted aryl or unsubstituted or substituted hetaryl, where C₁-C₁₂-alkyl may in each case be interrupted once or more than once, for example once, twice, thrice or more than thrice, by O or S.

R^{C} in the groups of the formula (III), in the compounds of the formula (II), is preferably hydrogen, C₁-C₁₂-alkyl, C₂-C₁₂-alkenyl or C₂-C₁₂-alkynyl, where the aforementioned radicals may in each case be interrupted once or more than once by O or S. More preferably, R^{C} is hydrogen or C₁-C₁₂-alkyl, where the carbon chain in C₁-C₁₂-alkyl is not interrupted by O or S. Most preferably, R^{C} is hydrogen or C₁-C₆-alkyl, where the carbon chain in C₁-C₆-alkyl is not interrupted by O or S.

In a specific embodiment of the present invention, in the compounds of the formula (II), R^{C} in the groups of the formula (III) is hydrogen.

Specific examples of the R^{A} and R^{B} radicals in the compounds of the formula (II) are:

1-ethylpropyl, 1-methylpropyl, 1-propylbutyl, 1-ethylbutyl, 1-methylbutyl, 1-butylpentyl, 1-propylpentyl, 1-ethylpentyl, 1-methylpentyl, 1-pentylhexyl, 1-butylhexyl, 1-propylhexyl, 1-ethylhexyl, 1-methylhexyl, 1-hexylheptyl, 1-pentylheptyl, 1-butylheptyl, 1-propylheptyl, 1-ethylheptyl, 1-methylheptyl, 1-heptyloctyl, 1-hexyloctyl, 1-pentyloctyl, 1-butyloctyl, 1-propyloctyl, 1-ethyloctyl, 1-methyloctyl, 1-octylnonyl, 1-heptylnonyl, 1-hexylnonyl, 1-pentylnonyl, 1-butylnonyl, 1-propylnonyl, 1-ethylnonyl, 1-methylnonyl, 1-nonyldecyl, 1-octyldecyl, 1-heptyldecyl, 1-hexyldecyl, 1-pentyldecyl, 1-butyldecyl, 1-propyldecyl, 1-ethyldecyl, 1-methyldecyl, 1-decylundecyl, 1-nonylundecyl, 1-octylundecyl, 1-heptylundecyl, 1-hexylundecyl, 1-pentylundecyl, 1-butylundecyl, 1-propylundecyl, 1-ethylundecyl, 1-methylundecyl, 1-undecyldodecyl, 1-decyldodecyl, 1-nonyldodecyl, 1-octyldodecyl, 1-heptyldodecyl, 1-hexyldodecyl, 1-pentyldodecyl, 1-butyldodecyl, 1-propyldodecyl, 1-ethyldodecyl, 1-methyldodecyl, 1-dodecyltridecyl, 1-undecyltridecyl, 1-decyltridecyl, 1-nonyltridecyl, 1-octyltridecyl, 1-heptyltridecyl, 1-hexyltridecyl, 1-pentyltridecyl, 1-butyltridecyl, 1-propyltridecyl, 1-ethyltridecyl, 1-methyltridecyl, 1-tridecyltetradecyl, 1-undecyltetradecyl, 1-decyltetradecyl, 1-nonyltetradecyl, 1-octyltetradecyl, 1-heptyltetradecyl, 1-hexyltetradecyl, 1-pentyltetradecyl, 1-butyltetradecyl, 1-propyltetradecyl, 1-ethyltetradecyl, 1-methyltetradecyl, 1-pentadecylhexadecyl, 1-tetradecylhexadecyl, 1-tridecylhexadecyl, 1-dodecylhexadecyl, 1-undecylhexadecyl, 1-decylhexadecyl, 1-nonylhexadecyl, 1-octylhexadecyl, 1-heptylhexadecyl, 1-hexylhexadecyl, 1-pentylhexadecyl, 1-butylhexadecyl, 1-propylhexadecyl, 1-ethylhexadecyl, 1-methylhexadecyl, 1-hexadecyloctadecyl, 1-pentadecyloctadecyl, 1-tetradecyloctadecyl, 1-tridecyloctadecyl, 1-dodecyloctadecyl, 1-undecyloctadecyl, 1-decyloctadecyl, 1-nonyloctadecyl, 1-octyloctadecyl, 1-heptyloctadecyl, 1-hexyloctadecyl, 1-pentyloctadecyl, 1-butyloctadecyl, 1-propyloctadecyl, 1-ethyloctadecyl, 1-methyloctadecyl, 1-nonadecyleicosanyl, 1-octadecyleicosanyl, 1-heptadecyleicosanyl, 1-hexadecyleicosanyl, 1-pentadecyleicosanyl, 1-tetradecyleicosanyl, 1-tridecyleicosanyl, 1-dodecyleicosanyl, 1-undecyleicosanyl, 1-decyleicosanyl, 1-nonyleicosanyl, 1-octyleicosanyl, 1-heptyleicosanyl, 1-hexyleicosanyl, 1-pentyleicosanyl, 1-butyleicosanyl, 1-propyleicosanyl, 1-ethyleicosanyl, 1-methyleicosanyl, 1-eicosanyldocosanyl, 1-nonadecyldocosanyl, 1-octadecyldocosanyl, 1-heptadecyldocosanyl, 1-hexadecyldocosanyl, 1-pentadecyldocosanyl, 1-tetradecyldocosanyl, 1-tridecyldocosanyl, 1-undecyldocosanyl, 1-decyldocosanyl, 1-nonyldocosanyl, 1-octyldocosanyl, 1-heptyldocosanyl, 1-hexyldocosanyl, 1-pentyldocosanyl, 1-butyldocosanyl, 1-propyldocosanyl, 1-ethyldocosanyl, 1-methyldocosanyl 1-tricosanyltetracosanyl, 1-docosanyltetracosanyl, 1-nonadecyltetracosanyl, 1-octadecyltetracosanyl, 1-heptadecyltetracosanyl, 1-hexadecyltetracosanyl, 1-pentadecyltetracosanyl, 1-pentadecyltetracosanyl, 1-tetradecyltetracosanyl, 1-tridecyltetracosanyl, 1-dodecyltetracosanyl, 1-undecyltetracosanyl, 1-decyltetracosanyl, 1-nonyltetracosanyl, 1-octyltetracosanyl, 1-heptyltetracosanyl, 1-hexyltetracosanyl, 1-pentyltetracosanyl, 1-butyltetracosanyl, 1-propyltetracosanyl, 1-ethyltetracosanyl, 1-methyltetracosanyl, 1-heptacosanyloctacosanyl, 1-hexacosanyloctacosanyl, 1-pentacosanyloctacosanyl, 1-tetracosanyloctacosanyl, 1-tricosanyloctacosanyl, 1-docosanyloctacosanyl, 1-nonadecyloctacosanyl, 1-octadecyloctacosanyl, 1-heptadecyloctacosanyl, 1-hexadecyloctacosanyl, 1-hexadecyloctacosanyl, 1-pentadecyloctacosanyl, 1-tetradecyloctacosanyl, 1-tridecyloctacosanyl, 1-dodecyloctacosanyl, 1-undecyloctacosanyl, 1-decyloctacosanyl, 1-nonyloctacosanyl, 1-octyloctacosanyl, 1-heptyloctacosanyl, 1-hexyloctacosanyl, 1-pentyloctacosanyl, 1-butyloctacosanyl, 1-propyloctacosanyl, 1-ethyloctacosanyl, 1-methyloctacosanyl and homologs thereof.

Particularly preferred examples of the R^{A} and R^{B} radicals in the compounds of the formula (II) 1,2,2′-tribranched alkyl radicals. These are specifically:
1-(1-methylethyl)-2-methylpropyl, 1-(1-methylethyl)-2-methylbutyl, 1-(1-methylpropyl)-2-methylbutyl, 1-(1-ethylpropyl)-2-methylbutyl, 1-(1-methylpropyl)-2-ethylbutyl, 1-(1-ethylpropyl)-2-ethylbutyl, 1-(1-methylethyl)-2-methylpentyl, 1-(1-methylpropyl)-2-methylpentyl, 1-(1-ethylpropyl)-2-methylpentyl, 1-(1-methylpropyl)-2-ethylpentyl, 1-(1-ethylpropyl)-2-ethylpentyl, 1-(1-methylbutyl)-2-methylpentyl, 1-(1-ethylbutyl)-2-methylpentyl, 1-(1-propylbutyl)-2-methylpentyl, 1-(1-methylbutyl)-2-ethylpentyl, 1-(1-ethylbutyl)-2-ethylpentyl, 1-(1-propylbutyl)-2-ethylpentyl, 1-(1-methylbutyl)-2-propylpentyl, 1-(1-ethylbutyl)-2-propylpentyl, 1-(1-propylbutyl)-2-propylpentyl, 1-(1-methylethyl)-2-methylhexyl, 1-(1-methylpropyl)-2-methylhexyl, 1-(1-ethylpropyl)-2-methylhexyl, 1-(1-methylpropyl)-2-ethylhexyl, 1-(1-ethylpropyl)-2-ethylhexyl, 1-(1-methylbutyl)-2-methylhexyl, 1-(1-ethylbutyl)-2-methylhexyl, 1-(1-propylbutyl)-2-methylhexyl, 1-(1-methylbutyl)-2-ethylhexyl, 1-(1-ethylbutyl)-2-ethylhexyl, 1-(1-propylbutyl)-2-ethylhexyl, 1-(1-methylbutyl)-2-propylhexyl, 1-(1-ethylbutyl)-2-propylhexyl, 1-(1-propylbutyl)-2-propylhexyl, 1-(1-methylpentyl)-2-methylhexyl, 1-(1-ethylpentyl)-2-methylhexyl, 1-(1-propylpentyl)-2-methylhexyl, 1-(1-butylpentyl)-2-methylhexyl, 1-(1-methylpentyl)-2-ethylhexyl, 1-(1-ethylpentyl)-2-ethylhexyl, 1-(1-propylpentyl)-2-ethylhexyl, 1-(1-butylpentyl)-2-ethylhexyl, 1-(1-methylpentyl)-2-propylhexyl, 1-(1-ethylpentyl)-2-propylhexyl, 1-(1-propylpentyl)-2-propylhexyl, 1-(1-butylpentyl)-2-propylhexyl, 1-(1-methylpentyl)-2-butylhexyl, 1-(1-ethylpentyl)-2-butylhexyl, 1-(1-propylpentyl)-2-butylhexyl, 1-(1-butylpentyl)-2-butylhexyl, 1-(1-methylethyl)-2-methylheptyl, 1-(1-methylpropyl)-2-methylheptyl, 1-(1-ethylpropyl)-2-methylheptyl, 1-(1-methylpropyl)-2-ethylheptyl, 1-(1-ethylpropyl)-2-ethylheptyl, 1-(1-methylbutyl)-2-methylheptyl, 1-(1-ethylbutyl)-2-methylheptyl, 1-(1-propylbutyl)-2-methylheptyl, 1-(1-methylbutyl)-2-ethylheptyl, 1-(1-ethylbutyl)-2-ethylheptyl, 1-(1-propylbutyl)-2-ethylheptyl, 1-(1-methylbutyl)-2-propylheptyl, 1-(1-ethylbutyl)-2-propylheptyl, 1-(1-propylbutyl)-2-propylheptyl, 1-(1-methylpentyl)-2-methylheptyl, 1-(1-ethylpentyl)-2-methylheptyl, 1-(1-propylpentyl)-2-methylheptyl, 1-(1-butylpentyl)-2-methylheptyl, 1-(1-methylpentyl)-2-ethylheptyl, 1-(1-ethylpentyl)-2-ethylheptyl, 1-(1-propylpentyl)-2-ethylheptyl, 1-(1-butylpentyl)-2-ethylheptyl, 1-(1-methylpentyl)-2-propylheptyl, 1-(1-ethylpentyl)-2-propylheptyl, 1-(1-propylpentyl)-2-propylheptyl, 1-(1-butylpentyl)-2-propylheptyl, 1-(1-methylpentyl)-2-butylheptyl, 1-(1-ethylpentyl)-2-butylheptyl, 1-(1-propylpentyl)-2-butylheptyl, 1-(1-butylpentyl)-2-butylheptyl, 1-(1-methylhexyl)-2-methylheptyl, 1-(1-ethylhexyl)-2-methylheptyl, 1-(1-propylhexyl)-2-methylheptyl, 1-(1-butylhexyl)-2-methylheptyl, 1-(1-pentylhexyl)-2-methylheptyl, 1-(1-methylhexyl)-2-ethylheptyl, 1-(1-ethylhexyl)-2-ethylheptyl, 1-(1-propylhexyl)-2-ethylheptyl, 1-(1-butylhexyl)-2-ethylheptyl, 1-(1-pentylhexyl)-2-ethylheptyl, 1-(1-methylhexyl)-2-propylheptyl, 1-(1-ethylhexyl)-2-propylheptyl, 1-(1-propylhexyl)-2-propylheptyl, 1-(1-butylhexyl)-2-propylheptyl, 1-(1-pentylhexyl)-2-propylheptyl, 1-(1-methylhexyl)-2-butylheptyl, 1-(1-ethylhexyl)-2-butylheptyl, 1-(1-propylhexyl)-2-butylheptyl, 1-(1-butylhexyl)-2-butylheptyl, 1-(1-pentylhexyl)-2-butylheptyl, 1-(1-methylhexyl)-2-pentylheptyl, 1-(1-ethylhexyl)-2-pentylheptyl, 1-(1-propylhexyl)-2-pentylheptyl, 1-(1-butylhexyl)-2-pentylheptyl, 1-(1-pentylhexyl)-2-pentylheptyl, 1-(1-methylethyl)-2-methyloctyl, 1-(1-methylpropyl)-2-methyloctyl, 1-(1-ethylpropyl)-2-methyloctyl, 1-(1-methylpropyl)-2-ethyloctyl, 1-(1-ethylpropyl)-2-ethyloctyl, 1-(1-methylbutyl)-2-methyloctyl, 1-(1-ethylbutyl)-2-methyloctyl, 1-(1-propylbutyl)-2-methyloctyl, 1-(1-methylbutyl)-2-ethyloctyl, 1-(1-ethylbutyl)-2-ethyloctyl, 1-(1-propylbutyl)-2-ethyloctyl, 1-(1-methylbutyl)-2-propyloctyl, 1-(1-ethylbutyl)-2-propyloctyl, 1-(1-propylbutyl)-2-propyloctyl, 1-(1-methylpentyl)-2-methyloctyl, 1-(1-ethylpentyl)-2-methyloctyl, 1-(1-propylpentyl)-2-methyloctyl, 1-(1-butylpentyl)-2-methyloctyl, 1-(1-methylpentyl)-2-ethyloctyl, 1-(1-ethylpentyl)-2-ethyloctyl, 1-(1-propylpentyl)-2-ethyloctyl, 1-(1-butylpentyl)-2-ethyloctyl, 1-(1-methylpentyl)-2-propyloctyl, 1-(1-ethylpentyl)-2-propyloctyl, 1-(1-propylpentyl)-2-propyloctyl, 1-(1-butylpentyl)-2-propyloctyl, 1-(1-methylpentyl)-2-butyloctyl, 1-(1-ethylpentyl)-2-butyloctyl, 1-(1-propylpentyl)-2-butyloctyl, 1-(1-butylpentyl)-2-butyloctyl, 1-(1-methylhexyl)-2-methyloctyl, 1-(1-ethylhexyl)-2-methyloctyl, 1-(1-propylhexyl)-2-methyloctyl, 1-(1-butylhexyl)-2-methyloctyl, 1-(1-pentylhexyl)-2-methyloctyl, 1-(1-methylhexyl)-2-ethyloctyl, 1-(1-ethylhexyl)-2-ethyloctyl, 1-(1-propylhexyl)-2-ethyloctyl, 1-(1-butylhexyl)-2-ethyloctyl, 1-(1-pentylhexyl)-2-ethyloctyl, 1-(1-methylhexyl)-2-propyloctyl, 1-(1-ethylhexyl)-2-propyloctyl, 1-(1-propylhexyl)-2-propyloctyl, 1-(1-butylhexyl)-2-propyloctyl, 1-(1-pentylhexyl)-2-propyloctyl, 1-(1-methylhexyl)-2-butyloctyl, 1-(1-ethylhexyl)-2-butyloctyl, 1-(1-propylhexyl)-2-butyloctyl, 1-(1-butylhexyl)-2-butyloctyl, 1-(1-pentylhexyl)-2-butyloctyl, 1-(1-methylhexyl)-2-pentyloctyl, 1-(1-ethylhexyl)-2-pentyloctyl, 1-(1-propylhexyl)-2-pentyloctyl, 1-(1-butylhexyl)-2-pentyloctyl, 1-(1-pentylhexyl)-2-pentyloctyl, 1-(1-methylheptyl)-2-methyloctyl, 1-(1-ethylheptyl)-2-methyloctyl, 1-(1-propylheptyl)-2-methyloctyl, 1-(1-butylheptyl)-2-methyloctyl, 1-(1-pentylheptyl)-2-methyloctyl, 1-(1-hexylheptyl)-2-methyloctyl, 1-(1-methylheptyl)-2-ethyloctyl, 1-(1-ethylheptyl)-2-ethyloctyl, 1-(1-propylheptyl)-2-ethyloctyl, 1-(1-butylheptyl)-2-ethyloctyl, 1-(1-pentylheptyl)-2-ethyloctyl, 1-(1-hexylheptyl)-2-ethyloctyl, 1-(1-methylheptyl)-2-propyloctyl, 1-(1-ethylheptyl)-2-propyloctyl, 1-(1-propylheptyl)-2-propyloctyl, 1-(1-butylheptyl)-2-propyloctyl, 1-(1-pentylheptyl)-2-propyloctyl, 1-(1-hexylheptyl)-2-propyloctyl, 1-(1-methylheptyl)-2-butyloctyl, 1-(1-ethylheptyl)-2-butyloctyl, 1-(1-propylheptyl)-2-butyloctyl, 1-(1-butylheptyl)-2-butyloctyl, 1-(1-pentylheptyl)-2-butyloctyl, 1-(1-hexylheptyl)-2-butyloctyl, 1-(1-methylheptyl)-2-pentyloctyl, 1-(1-ethylheptyl)-2-pentyloctyl, 1-(1-propylheptyl)-2-pentyloctyl, 1-(1-butylheptyl)-2-pentyloctyl, 1-(1-pentylheptyl)-2-pentyloctyl, 1-(1-hexylheptyl)-2-pentyloctyl, 1-(1-methylheptyl)-2-hexyloctyl, 1-(1-ethylheptyl)-2-hexyloctyl, 1-(1-propylheptyl)-2-hexyloctyl, 1-(1-butylheptyl)-2-hexyloctyl, 1-(1-pentylheptyl)-2-hexyloctyl, 1-(1-hexylheptyl)-2-hexyloctyl, 1-(1-methylethyl)-2-methylnonyl, 1-(1-methylpropyl)-2-methylnonyl, 1-(1-ethylpropyl)-2-methylnonyl, 1-(1-methylpropyl)-2-ethylnonyl, 1-(1-ethylpropyl)-2-ethylnonyl, 1-(1-methylbutyl)-2-methylnonyl, 1-(1-ethylbutyl)-2-methylnonyl, 1-(1-propylbutyl)-2-methylnonyl, 1-(1-methylbutyl)-2-ethylnonyl, 1-(1-ethylbutyl)-2-ethylnonyl, 1-(1-propylbutyl)-2-ethylnonyl, 1-(1-methylbutyl)-2-propylnonyl, 1-(1-ethylbutyl)-2-propylnonyl, 1-(1-propylbutyl)-2-propylnonyl, 1-(1-methylpentyl)-2-methylnonyl, 1-(1-ethylpentyl)-2-methylnonyl, 1-(1-propylpentyl)-2-methylnonyl, 1-(1-butylpentyl)-2-methylnonyl, 1-(1-methylpentyl)-2-ethylnonyl, 1-(1-ethylpentyl)-2-ethylnonyl, 1-(1-propylpentyl)-2-ethylnonyl, 1-(1-butylpentyl)-2-ethylnonyl, 1-(1-methylpentyl)-2-propylnonyl, 1-(1-ethylpentyl)-2-propylnonyl, 1-(1-propylpentyl)-2-propylnonyl, 1-(1-butylpentyl)-2-propylnonyl, 1-(1-methylpentyl)-2-butylnonyl, 1-(1-ethylpentyl)-2-butylnonyl, 1-(1-propylpentyl)-2-butylnonyl, 1-(1-butylpentyl)-2-butylnonyl, 1-(1-methylhexyl)-2-methylnonyl, 1-(1-ethylhexyl)-2-methylnonyl, 1-(1-propylhexyl)-2-methylnonyl, 1-(1-butylhexyl)-2-methylnonyl, 1-(1-pentylhexyl)-2-methylnonyl, 1-(1-methylhexyl)-2-ethylnonyl, 1-(1-ethylhexyl)-2-ethylnonyl, 1-(1-propylhexyl)-2-ethylnonyl, 1-(1-butylhexyl)-2-ethylnonyl, 1-(1-pentylhexyl)-2-ethylnonyl, 1-(1-methylhexyl)-2-propylnonyl, 1-(1-ethylhexyl)-2-propylnonyl, 1-(1-propylhexyl)-2-propylnonyl, 1-(1-butylhexyl)-2-propylnonyl, 1-(1-pentylhexyl)-2-propylnonyl, 1-(1-methylhexyl)-2-butylnonyl, 1-(1-ethylhexyl)-2-butylnonyl, 1-(1-propylhexyl)-2-butylnonyl, 1-(1-butylhexyl)-2-butylnonyl, 1-(1-pentylhexyl)-2-butylnonyl, 1-(1-methylhexyl)-2-pentylnonyl, 1-(1-ethylhexyl)-2-pentylnonyl, 1-(1-propylhexyl)-2-pentylnonyl, 1-(1-butylhexyl)-2-pentylnonyl, 1-(1-pentylhexyl)-2-pentylnonyl, 1-(1-methylheptyl)-2-methylnonyl, 1-(1-ethylheptyl)-2-methylnonyl, 1-(1-propylheptyl)-2-methylnonyl, 1-(1-butylheptyl)-2-methylnonyl, 1-(1-pentylheptyl)-2-methylnonyl, 1-(1-hexylheptyl)-2-methylnonyl, 1-(1-methylheptyl)-2-ethylnonyl, 1-(1-ethylheptyl)-2-ethylnonyl, 1-(1-propylheptyl)-2-ethylnonyl, 1-(1-butylheptyl)-2-ethylnonyl, 1-(1-pentylheptyl)-2-ethylnonyl, 1-(1-hexylheptyl)-2-ethylnonyl, 1-(1-methylheptyl)-2-propylnonyl, 1-(1-ethylheptyl)-2-propylnonyl, 1-(1-propylheptyl)-2-propylnonyl, 1-(1-butylheptyl)-2-propylnonyl, 1-(1-pentylheptyl)-2-propylnonyl, 1-(1-hexylheptyl)-2-propylnonyl, 1-(1-methylheptyl)-2-butylnonyl, 1-(1-ethylheptyl)-2-butylnonyl, 1-(1-propylheptyl)-2-butylnonyl, 1-(1-butylheptyl)-2-butylnonyl, 1-(1-pentylheptyl)-2-butylnonyl, 1-(1-hexylheptyl)-2-butylnonyl, 1-(1-methylheptyl)-2-pentylnonyl, 1-(1-ethylheptyl)-2-pentylnonyl, 1-(1-propylheptyl)-2-pentylnonyl, 1-(1-butylheptyl)-2-pentylnonyl, 1-(1-pentylheptyl)-2-pentylnonyl, 1-(1-hexylheptyl)-2-pentylnonyl, 1-(1-methylheptyl)-2-hexylnonyl, 1-(1-ethylheptyl)-2-hexylnonyl, 1-(1-propylheptyl)-2-hexylnonyl, 1-(1-butylheptyl)-2-hexylnonyl, 1-(1-pentylheptyl)-2-hexylnonyl, 1-(1-hexyl heptyl)-2-hexylnonyl, 1-(1-methyloctyl)-2-methylnonyl, 1-(1-ethyloctyl)-2-methylnonyl, 1-(1-propyloctyl)-2-methylnonyl, 1-(1-butyloctyl)-2-methylnonyl, 1-(1-pentyloctyl)-2-methylnonyl, 1-(1-hexyloctyl)-2-methylnonyl, 1-(1-heptyloctyl)-2-methylnonyl, 1-(1-methyloctyl)-2-ethylnonyl, 1-(1-ethyloctyl)-2-ethylnonyl, 1-(1-propyloctyl)-2-ethylnonyl, 1-(1-butyloctyl)-2-ethylnonyl, 1-(1-pentyloctyl)-2-ethylnonyl, 1-(1-hexyloctyl)-2-ethylnonyl, 1-(1-heptyloctyl)-2-ethylnonyl, 1-(1-methyloctyl)-2-propylnonyl, 1-(1-ethyloctyl)-2-propylnonyl, 1-(1-propyloctyl)-2-propylnonyl, 1-(1-butyloctyl)-2-propylnonyl, 1-(1-pentyloctyl)-2-propylnonyl, 1-(1-hexyloctyl)-2-propylnonyl, 1-(1-heptyloctyl)-2-propylnonyl, 1-(1-methyloctyl)-2-butylnonyl, 1-(1-ethyloctyl)-2-butylnonyl, 1-(1-propyloctyl)-2-butylnonyl, 1-(1-butyloctyl)-2-butylnonyl, 1-(1-pentyloctyl)-2-butylnonyl, 1-(1-hexyloctyl)-2-butylnonyl, 1-(1-heptyloctyl)-2-butylnonyl, 1-(1-methyloctyl)-2-pentylnonyl, 1-(1-ethyloctyl)-2-pentylnonyl, 1-(1-propyloctyl)-2-pentylnonyl, 1-(1-butyloctyl)-2-pentylnonyl, 1-(1-pentyloctyl)-2-pentylnonyl, 1-(1-hexyloctyl)-2-pentylnonyl, 1-(1-heptyloctyl)-2-pentylnonyl, 1-(1-methyloctyl)-2-hexylnonyl, 1-(1-ethyloctyl)-2-hexylnonyl, 1-(1-propyloctyl)-2-hexylnonyl, 1-(1-butyloctyl)-2-hexylnonyl, 1-(1-pentyloctyl)-2-hexylnonyl, 1-(1-hexyloctyl)-2-hexylnonyl, 1-(1-heptyloctyl)-2-heptylnonyl, 1-(1-methyloctyl)-2-heptylnonyl, 1-(1-ethyloctyl)-2-heptylnonyl, 1-(1-propyloctyl)-2-heptylnonyl, 1-(1-butyloctyl)-2-heptylnonyl, 1-(1-pentyloctyl)-2-heptylnonyl, 1-(1-hexyloctyl)-2-heptylnonyl, 1-(1-heptyloctyl)-2-heptylnonyl, 1-(1-methylethyl)-2-methyldecyl, 1-(1-methylpropyl)-2-methyldecyl, 1-(1-ethylpropyl)-2-methyldecyl, 1-(1-methylpropyl)-2-ethyldecyl, 1-(1-ethylpropyl)-2-ethyldecyl, 1-(1-methylbutyl)-2-methyldecyl, 1-(1-ethylbutyl)-2-methyldecyl, 1-(1-propylbutyl)-2-methyldecyl, 1-(1-methylbutyl)-2-ethyldecyl, 1-(1-ethylbutyl)-2-ethyldecyl, 1-(1-propylbutyl)-2-ethyldecyl, 1-(1-methylbutyl)-2-propyldecyl, 1-(1-ethylbutyl)-2-propyldecyl, 1-(1-propylbutyl)-2-propyldecyl, 1-(1-methylpentyl)-2-methyldecyl, 1-(1-ethylpentyl)-2-methyldecyl, 1-(1-propylpentyl)-2-methyldecyl, 1-(1-butylpentyl)-2-methyldecyl, 1-(1-methylpentyl)-2-ethyldecyl, 1-(1-ethylpentyl)-2-ethyldecyl, 1-(1-propylpentyl)-2-ethyldecyl, 1-(1-butylpentyl)-2-ethyldecyl, 1-(1-methylpentyl)-2-propyldecyl, 1-(1-ethylpentyl)-2-propyldecyl, 1-(1-propylpentyl)-2-propyldecyl, 1-(1-butylpentyl)-2-propyldecyl, 1-(1-methylpentyl)-2-butyldecyl, 1-(1-ethylpentyl)-2-butyldecyl, 1-(1-propylpentyl)-2-butyldecyl, 1-(1-butylpentyl)-2-butyldecyl, 1-(1-methylhexyl)-2-methyldecyl, 1-(1-ethylhexyl)-2-methyldecyl, 1-(1-propylhexyl)-2-methyldecyl, 1-(1-butylhexyl)-2-methyldecyl, 1-(1-pentylhexyl)-2-methyldecyl, 1-(1-methylhexyl)-2-ethyldecyl, 1-(1-ethylhexyl)-2-ethyldecyl, 1-(1-propylhexyl)-2-ethyldecyl, 1-(1-butylhexyl)-2-ethyldecyl, 1-(1-pentylhexyl)-2-ethyldecyl, 1-(1-methylhexyl)-2-propyldecyl, 1-(1-ethylhexyl)-2-propyldecyl, 1-(1-propylhexyl)-2-propyldecyl, 1-(1-butylhexyl)-2-propyldecyl, 1-(1-pentylhexyl)-2-propyldecyl, 1-(1-methylhexyl)-2-butyldecyl, 1-(1-ethylhexyl)-2-butyldecyl, 1-(1-propylhexyl)-2-butyldecyl, 1-(1-butylhexyl)-2-butyldecyl, 1-(1-pentylhexyl)-2-butyldecyl, 1-(1-methylhexyl)-2-pentyldecyl, 1-(1-ethylhexyl)-2-pentyldecyl, 1-(1-propylhexyl)-2-pentyldecyl, 1-(1-butylhexyl)-2-pentyldecyl, 1-(1-pentylhexyl)-2-pentyldecyl, 1-(1-methylheptyl)-2-methyldecyl, 1-(1-ethylheptyl)-2-methyldecyl, 1-(1-propylheptyl)-2-methyldecyl, 1-(1-butylheptyl)-2-methyldecyl, 1-(1-pentylheptyl)-2-methyldecyl, 1-(1-hexylheptyl)-2-methyldecyl, 1-(1-methylheptyl)-2-ethyldecyl, 1-(1-ethylheptyl)-2-ethyldecyl, 1-(1-propylheptyl)-2-ethyldecyl, 1-(1-butylheptyl)-2-ethyldecyl, 1-(1-pentylheptyl)-2-ethyldecyl, 1-(1-hexylheptyl)-2-ethyldecyl, 1-(1-methylheptyl)-2-propyldecyl, 1-(1-ethylheptyl)-2-propyldecyl, 1-(1-propylheptyl)-2-propyldecyl, 1-(1-butylheptyl)-2-propyldecyl, 1-(1-pentylheptyl)-2-propyldecyl, 1-(1-hexylheptyl)-2-propyldecyl, 1-(1-methylheptyl)-2-butyldecyl, 1-(1-ethylheptyl)-2-butyldecyl, 1-(1-propylheptyl)-2-butyldecyl, 1-(1-butylheptyl)-2-butyldecyl, 1-(1-pentylheptyl)-2-butyldecyl, 1-(1-hexylheptyl)-2-butyldecyl, 1-(1-methylheptyl)-2-pentyldecyl, 1-(1-ethylheptyl)-2-pentyldecyl, 1-(1-propylheptyl)-2-pentyldecyl, 1-(1-butylheptyl)-2-pentyldecyl, 1-(1-pentylheptyl)-2-pentyldecyl, 1-(1-hexylheptyl)-2-pentyldecyl, 1-(1-methylheptyl)-2-hexyldecyl, 1-(1-ethylheptyl)-2-hexyldecyl, 1-(1-propylheptyl)-2-hexyldecyl, 1-(1-butylheptyl)-2-hexyldecyl, 1-(1-pentylheptyl)-2-hexyldecyl, 1-(1-hexylheptyl)-2-hexyldecyl, 1-(1-methyloctyl)-2-methyldecyl, 1-(1-ethyloctyl)-2-methyldecyl, 1-(1-propyloctyl)-2-methyldecyl, 1-(1-butyloctyl)-2-methyldecyl, 1-(1-pentyloctyl)-2-methyldecyl, 1-(1-hexyloctyl)-2-methyldecyl, 1-(1-heptyloctyl)-2-methyldecyl, 1-(1-methyloctyl)-2-ethyldecyl, 1-(1-ethyloctyl)-2-ethyldecyl, 1-(1-propyloctyl)-2-ethyldecyl, 1-(1-butyloctyl)-2-ethyldecyl, 1-(1-pentyloctyl)-2-ethyldecyl, 1-(1-hexyloctyl)-2-ethyldecyl, 1-(1-heptyloctyl)-2-ethyldecyl, 1-(1-methyloctyl)-2-propyldecyl, 1-(1-ethyloctyl)-2-propyldecyl, 1-(1-propyloctyl)-2-propyldecyl, 1-(1-butyloctyl)-2-propyldecyl, 1-(1-pentyloctyl)-2-propyldecyl, 1-(1-hexyloctyl)-2-propyldecyl, 1-(1-heptyloctyl)-2-propyldecyl, 1-(1-methyloctyl)-2-butyldecyl, 1-(1-ethyloctyl)-2-butyldecyl, 1-(1-propyloctyl)-2-butyldecyl, 1-(1-butyloctyl)-2-butyldecyl, 1-(1-pentyloctyl)-2-butyldecyl, 1-(1-hexyloctyl)-2-butyldecyl, 1-(1-heptyloctyl)-2-butyldecyl, 1-(1-methyloctyl)-2-pentyldecyl, 1-(1-ethyloctyl)-2-pentyldecyl, 1-(1-propyloctyl)-2-pentyldecyl, 1-(1-butyloctyl)-2-pentyldecyl, 1-(1-pentyloctyl)-2-pentyldecyl, 1-(1-hexyloctyl)-2-pentyldecyl, 1-(1-heptyloctyl)-2-pentyldecyl, 1-(1-methyloctyl)-2-hexyldecyl, 1-(1-ethyloctyl)-2-hexyldecyl, 1-(1-propyloctyl)-2-hexyldecyl, 1-(1-butyloctyl)-2-hexyldecyl, 1-(1-pentyloctyl)-2-hexyldecyl, 1-(1-hexyloctyl)-2-hexyldecyl, 1-(1-heptyloctyl)-2-heptyldecyl, 1-(1-methyloctyl)-2-heptyldecyl, 1-(1-ethyloctyl)-2-heptyldecyl, 1-(1-propyloctyl)-2-heptyldecyl, 1-(1-butyloctyl)-2-heptyldecyl, 1-(1-pentyloctyl)-2-heptyldecyl, 1-(1-hexyloctyl)-2-heptyldecyl, 1-(1-heptyloctyl)-2-heptyldecyl, 1-(1-methylnonyl)-2-methyldecyl, 1-(1-ethylnonyl)-2-methyldecyl, 1-(1-propylnonyl)-2-methyldecyl, 1-(1-butylnonyl)-2-methyldecyl, 1-(1-pentylnonyl)-2-methyldecyl, 1-(1-hexylnonyl)-2-methyldecyl, 1-(1-heptylnonyl)-2-methyldecyl, 1-(1-octylnonyl)-2-methyldecyl, 1-(1-methylnonyl)-2-ethyldecyl, 1-(1-ethylnonyl)-2-ethyldecyl, 1-(1-propylnonyl)-2-ethyldecyl, 1-(1-butylnonyl)-2-ethyldecyl, 1-(1-pentylnonyl)-2-ethyldecyl, 1-(1-hexylnonyl)-2-ethyldecyl, 1-(1-heptylnonyl)-2-ethyldecyl, 1-(1-octylnonyl)-2-ethyldecyl, 1-(1-methylnonyl)-2-propyldecyl, 1-(1-ethylnonyl)-2-propyldecyl, 1-(1-propylnonyl)-2-propyldecyl, 1-(1-butylnonyl)-2-propyldecyl, 1-(1-pentylnonyl)-2-propyldecyl, 1-(1-hexylnonyl)-2-propyldecyl, 1-(1-heptylnonyl)-2-propyldecyl, 1-(1-octylnonyl)-2-propyldecyl, 1-(1-methylnonyl)-2-butyldecyl, 1-(1-ethylnonyl)-2-butyldecyl, 1-(1-propylnonyl)-2-butyldecyl, 1-(1-butylnonyl)-2-butyldecyl, 1-(1-pentylnonyl)-2-butyldecyl, 1-(1-hexylnonyl)-2-butyldecyl, 1-(1-heptylnonyl)-2-butyldecyl, 1-(1-octylnonyl)-2-butyldecyl, 1-(1-methylnonyl)-2-pentyldecyl, 1-(1-ethylnonyl)-2-pentyldecyl, 1-(1-propylnonyl)-2-pentyldecyl, 1-(1-butylnonyl)-2-pentyldecyl, 1-(1-pentylnonyl)-2-pentyldecyl, 1-(1-hexylnonyl)-2-pentyldecyl, 1-(1-heptylnonyl)-2-pentyldecyl, 1-(1-octylnonyl)-2-pentyldecyl, 1-(1-methylnonyl)-2-hexyldecyl, 1-(1-ethylnonyl)-2-hexyldecyl, 1-(1-propylnonyl)-2-hexyldecyl, 1-(1-butylnonyl)-2-hexyldecyl, 1-(1-pentylnonyl)-2-hexyldecyl, 1-(1-hexylnonyl)-2-hexyldecyl, 1-(1-heptylnonyl)-2-hexyldecyl, 1-(1-octylnonyl)-2-hexyldecyl, 1-(1-methylnonyl)-2-heptyldecyl, 1-(1-ethylnonyl)-2-heptyldecyl, 1-(1-propylnonyl)-2-heptyldecyl, 1-(1-butylnonyl)-2-heptyldecyl, 1-(1-pentylnonyl)-2-heptyldecyl, 1-(1-hexylnonyl)-2-heptyldecyl, 1-(1-heptylnonyl)-2-heptyldecyl, 1-(1-octylnonyl)-2-heptyldecyl, 1-(1-methylnonyl)-2-octyldecyl, 1-(1-ethylnonyl)-2-octyldecyl, 1-(1-propylnonyl)-2-octyldecyl, 1-(1-butylnonyl)-2-octyldecyl, 1-(1-pentylnonyl)-2-octyldecyl, 1-(1-hexylnonyl)-2-octyldecyl, 1-(1-heptylnonyl)-2-octyldecyl, 1-(1-octylnonyl)-2-octyldecyl, 1-(1-nonyldecyl)-2-nonylundecyl, 1-(1-decylundecyl)-2-decyldodecyl, 1-(1-undecyldodecyl)-2-undecyltridecyl, 1-(1-dodecyltridecyl)-2-dodecyltetradecyl and homologs thereof.

Very particularly preferred R^{A} and R^{B} radicals are 1-(1-methylethyl)-2-methylpropyl, 1-(1-ethylpropyl)-2-ethylbutyl, 1-(1-propylbutyl)-2-propylpentyl, 1-(1-butylpentyl)-2-butylhexyl, 1-(1-pentylhexyl)-2-pentylheptyl, 1-(1-hexylheptyl)-2-hexyloctyl, 1-(1-heptyloctyl)-2-heptylnonyl, 1-(1-octylnonyl)-2-octyldecyl, 1-(1-nonyldecyl)-2-nonylundecyl, 1-(1-decylundecyl)-2-decyldodecyl, 1-(1-undecyldodecyl)-2-undecyltridecyl and 1-(1-dodecyltridecyl)-2-dodecyltetradecyl.

For the use of the process according to the invention for producing field-effect transistors and solar cells, the compounds shown below, for example, are particularly suitable:

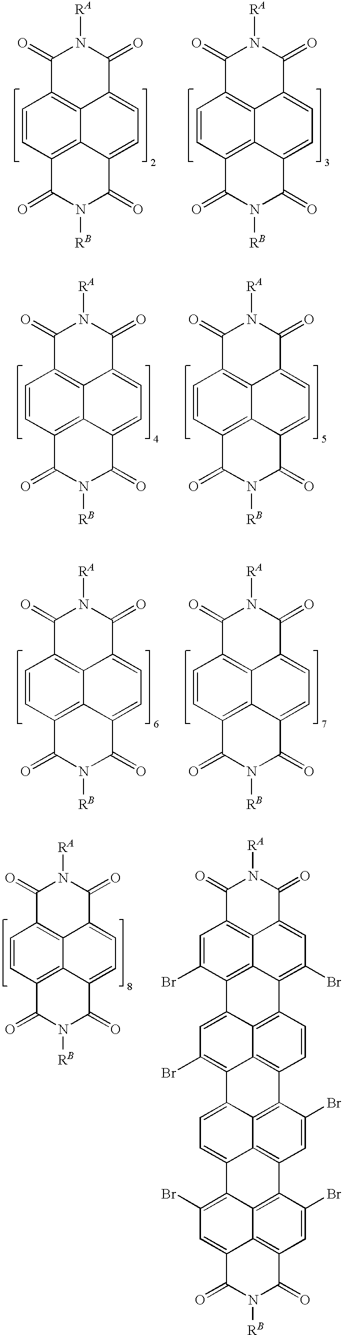

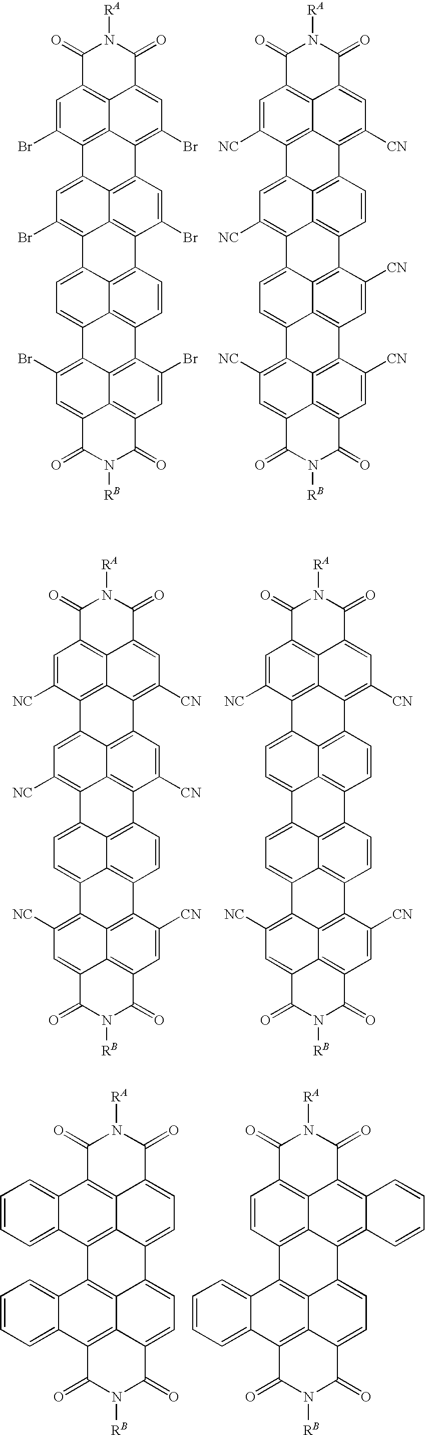

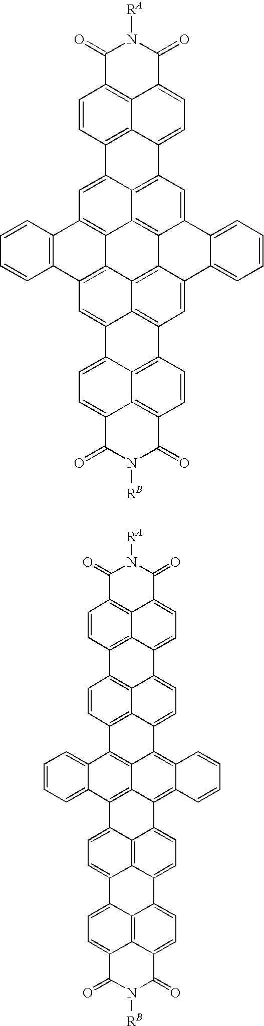

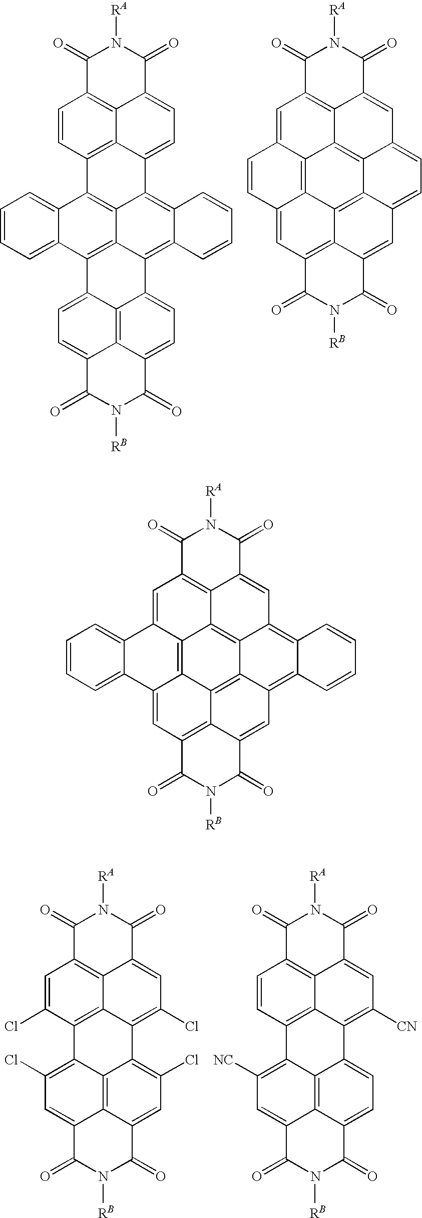

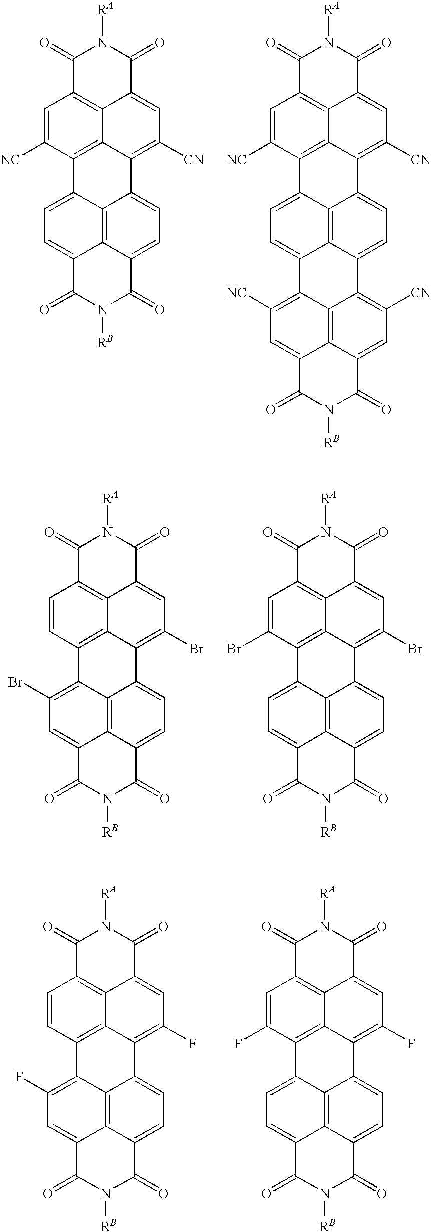

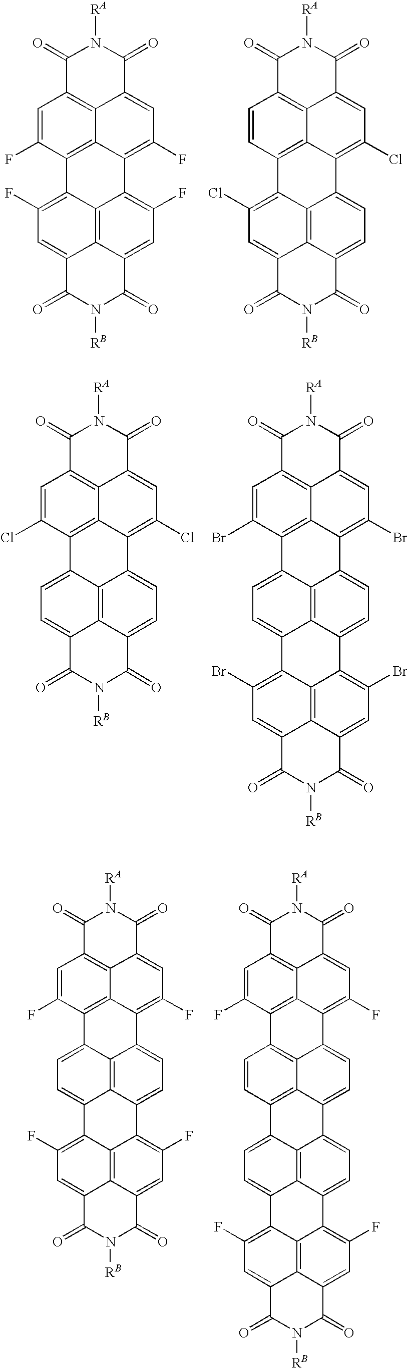

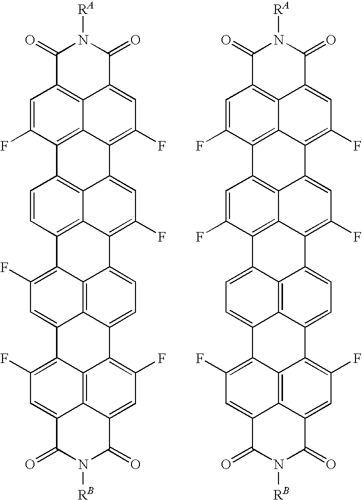

With regard to the preferred definitions of the R^{A} and R^{B} groups, reference is made to the above remarks.

Compounds of the formula (II) in which Rⁿ¹, Rⁿ², Rⁿ³ and Rⁿ⁴ are each independently hydrogen, F, Cl, Br, CN, alkoxy, alkylthio, alkylamino, dialkylamino, aryloxy, arylthio, hetaryloxy or hetarylthio are known or can be prepared analogously to processes known per se (see, for example, PCT/EP2007/053330; Chem. Mater. 2006, 18, 3715-3725; DE 10233955; DE 102004024909; Angew. Chem. (Int. Ed. Engl.) 2005, 117, 2479-2480; DE 102005018231; Angew. Chem. (Int. Ed. Engl.) 2006, 118, 1401-1404).

Compounds of the general formula (II) in which in each case two of the Rⁿ¹ and Rⁿ² and/or Rⁿ³ and Rⁿ⁴ radicals together are part of an aromatic ring system fused to one or two adjacent naphthalene units of the rylene skeleton (also known as coronenes) are likewise known per se or can be provided analogously to known processes (see, for example, Müllen, J. Mater. Chem., 11, 1789 (2001)).

### Step ii)

According to the invention, the substrate is treated with a solution of the compound of the formula (II) to obtain a thin layer of the compound of the formula (II) on the substrate. Thin layers of the compounds of the formula (II) can be produced by solution-processible methods such as spin-coating, knife-coating, casting methods, spray application, dip-coating or printing (e.g. inkjet, flexographic, offset, gravure; intaglio, nanoimprinting). Preference is given to those methods in which the production of the layers comprises an introduction of shear energy. The resulting layer thicknesses in this case are from 10 to 1000 nm, preferably from 10 to 400 nm. The resulting semiconductor layers thus generally have a thickness which is sufficient for ohmic content, for example, between source and drain electrode.

Preferred solvents for the inventive use of the compounds of the formula (II) are aromatic solvents such as benzene, toluene, xylene, mesitylene, chlorobenzene or dichlorobenzene, trialkylamines, nitrogen-containing heterocycles, N,N-disubstituted aliphatic carboxamides such as dimethylformamide, diethylformamide, dimethylacetamide or dimethylbutyramide, N-alkyllactams such as N-methyl-pyrrolidone, linear and cyclic ketones such as methyl ethyl ketone, cyclopentanone or cyclohexanone, cyclic ethers such as tetrahydrofuran or dioxane, esters such as ethyl acetate, butyl acetate, halogenated hydrocarbons such as chloroform or dichloromethane, and also mixtures of the solvents mentioned, which may additionally comprise alcohols such as methanol, ethanol, propanol, isopropanol or butanol.

The compounds of the formula (II) can be deposited on the substrate under an inert atmosphere, for example under nitrogen, argon or helium.

The deposition is effected typically within a pressure range from 0.5 to 1.5 bar. In particular, the deposition is effected at ambient pressure.

In a specific embodiment of the process according to the invention, the substrate is additionally treated with a thermally stable, electron-rich compound which is suitable for doping the layer of the compounds of the formula (I), or with a compound which is converted to such an electron-rich compound under the conditions of the heating in step iii). Such compounds which are typically used as dopants for semiconductors are known to those skilled in the art. Suitable examples thereof are pyronin B or rhodamine.

In a preferred embodiment of the process according to the invention, at least one compound of the general formula (II) (and if appropriate further semiconductor materials and/or dopants) is deposited by spin-coating or by printing.

In addition, the compound of the formula (II) is preferably deposited while introducing shear energy. This includes the typical knife-coating methods such as airknife-coating, knife-coating, airblade-coating, squeeze-coating, roll-coating and kiss-coating. For this purpose, for example, a solution of the compound of the formula (II) is applied to a first substrate and then a second substrate is brought into contact with the compound. Then shear energy is introduced. The shear rate is typically in the range from 0.04 to 30 mm/s and more typically from 0.4 to 3 mm/s. It may be advantageous to hydrophobize the surface of the second substrate. Suitable compounds for hydrophobizing substrate surfaces comprise alkyltrialkoxysilanes, such as n-octadecyltrimethoxysilane, n-octadecyltriethoxysilane, n-octadecyltri(n-propyl)-oxysilane or n-octadecyltri(isopropyl)oxysilane.

In a specific embodiment of the process according to the invention, the substrates obtained in step ii) are dried at temperatures in the range from room temperature to temperatures below 200° C., before the substrate is subjected to step iii). It may be advantageous to perform the drying under reduced pressure, for example in a pressure range from 10⁻³ to 1 bar, preferably from 10⁻² to 1 bar.

The drying time depends on the compounds of the formula (II) used in the specific case and the solvent used. In general, the drying will be carried out over a period from 10 seconds to 24 hours, preferably from 10 seconds to 16 hours, especially from 10 seconds to 8 hours and most preferably from 10 seconds to 1 hour.

Step iii)

According to the invention, the substrate treated with a compound of the formula (II) is heated in step iii) of the process according to the invention to a temperature at which at least some of the compounds of the formula (II) are converted to compounds of the formula (I). Typically, the substrate treated with a compound of the formula (II) will be heated in step iii) of the process according to the invention to a temperature in the range from 200 to 600° C., preferably from 250 to 550° C. and more preferably from 300 to 500° C., in order to bring about a defined, full conversion of the compounds of the formula (II) to the corresponding compounds of the formula (I).

Suitable apparatus for the heating of the compounds of the formula (II) is known to those skilled in the art. It is specifically apparatus which is typically used to dry and cure coatings, for example lacquers. The heat can be transferred by thermal radiation, thermal conduction or convection.

The heating time required for the conversion of the compounds of the formula (II) to compounds of the formula (I) depends on the compounds used in the individual case and can be determined in the individual case, for example, by thermogravimetry studies. Typically, the substrate coated with a compound of the formula (II) will only be heated for as long as is necessary for the conversion of the compounds of the formula (II) to compounds of the formula (I). However, it may, for example, also be advantageous to bring about longer-lasting heating of the substrate, in order to be able to carry out decomposition of the compounds of the formula (II) at lower temperatures. The duration of the heating will typically be within a range from one second up to 10 hours. After the conversion of the compounds of the formula (II) to compounds of the formula (I), the coated substrate can be subjected to a so-called “annealing” step, i.e. the coated substrate is heat-treated.

The compounds of the formula (II) can be thermolyzed under an inert atmosphere, for example under nitrogen, argon or helium.

The compounds of the formula (II) can be thermolyzed at ambient pressure or under the action of pressure.

In a preferred embodiment of the process according to the invention, the heat treatment of the substrate obtained in step ii) is effected under the action of pressure. Preferred pressure ranges are in the range from +5 to +80 kPa gauge, especially from +10 to +70 kPa gauge and most preferably from +20 to +60 kPa gauge. In the context of the invention, “kPa gauge” is understood to mean the difference between the absolute pressure and the existing atmospheric pressure, the absolute pressure being above the atmospheric pressure.

Suitable apparatus for performing the thermolysis under pressure is known in principle to those skilled in the art. Suitable apparatus for this purpose includes flat structures which are present on the substrate obtained in step ii) during the thermolysis. The weight of the flat structure may be sufficient to generate the elevated pressure, or an additional force is exerted on the flat structure. The additional force can act on the flat structure, for example, through weights or devices such as presses. The flat structure is preferably very regular with regard to its thickness and its weight over its area, and corresponds in terms of its circumference at least to that of the substrate. An example of a suitable flat structure is a plate, for example a glass plate.

The influence of pressure has a surprisingly positive effect on the quality of the resulting film. In an OFET, compounds of the formula (I) which have been obtained in step iii) by thermolysis of compounds of the formula (II) under the action of pressure have a significantly higher charge mobility compared to compounds of the formula (I) which have been obtained by thermolysis of the compounds of the formula (II) at ambient pressure.

In a specific embodiment of the process according to the invention, at least one compound of the general formula (II) (and if appropriate further semiconductor materials and/or dopants) is/are deposited in step ii) by introduction of shear energy and especially by shearing, and the thermolysis in step iii) is effected under the action of pressure. This specific embodiment of the process according to the invention enables higher charge mobilities of the compounds of the formula (I).

The coated substrate can be freed of the organic residues eliminated in the conversion of the compounds of the formula (II) to compounds of the formula (I), for example, under reduced pressure, specifically from 10⁻³ to 0.5 bar, and/or at a temperature from 150 to 600° C.

The compounds of PCT/EP 2008/053063 can be processed advantageously by the process according to the invention.

The invention further relates to the coated substrates obtainable by the process according to the invention.

The invention relates specifically to an inventive coated substrate comprising at least one compound of the formula (I) as emitter materials, charge transport materials or exciton transport materials.

Suitable substrates are in principle the materials known for this purpose. Suitable substrates comprise, for example, metals (preferably metals of groups 8, 9, 10 or 11 of the Periodic Table, such as Au, Ag, Cu), oxidic materials (such as glass, ceramics, SiO₂, especially quartz), semiconductors (e.g. doped Si, doped Ge), metal alloys (for example based on Au, Ag, Cu, etc.), semiconductor alloys, polymers (e.g. polyvinyl chloride, polyolefins such as polyethylene and polypropylene, polyesters, fluoropolymers, polyamides, polyimides, polyurethanes, polyalkyl (meth)acrylates, polystyrene and mixtures and composites thereof, inorganic solids (e.g. ammonium chloride), paper and combinations thereof. The substrates may be flexible or inflexible and have planar or curved geometry depending on the desired use.

A typical substrate for semiconductor units comprises a matrix (e.g. a quartz or polymer matrix) and, optionally, a dielectric top layer.

Suitable dielectrics are anodized aluminum (Al₂O₃), SiO₂, polystyrene, poly-α-methylstyrene, polyolefins (such as polypropylene, polyethylene, polyisobutene), polyvinylcarbazole, fluorinated polymers (e.g. Cytop), cyanopullulans (e.g. CYMM), polyvinylphenol, poly-p-xylene, polyvinyl chloride, or polymers crosslinkable thermally or by atmospheric moisture.

Specific dielectrics are “self-assembled nanodielectrics”, i.e. polymers which are obtained from monomers comprising SiCl functionalities, for example Cl₃SiOSiCl₃, Cl₃Si—(CH₂)₆—SiCl₃, Cl₃Si—(CH₂)₁₂—SiCl₃, and/or which are crosslinked by atmospheric moisture or by addition of water diluted with solvents (see, for example, Faccietti Adv. Mat. 2005, 17, 1705-1725). Instead of water, it is also possible for hydroxyl-containing polymers such as polyvinyl phenol or polyvinyl alcohol or copolymers of vinylphenol and styrene to serve as crosslinking components. It is also possible for at least one further polymer to be present during the crosslinking operation, for example polystyrene, which is then also crosslinked (see Facietti, US patent application 2006/0202195).

The substrate may additionally have electrodes, such as gate, drain and source electrodes of OFETs, which are normally localized on the substrate (for example deposited onto or embedded into a nonconductive layer on the dielectric). The substrate may additionally comprise conductive gate electrodes of the OFETs, which are typically arranged below the dielectric top layer (i.e. the gate dielectric).

The layer thicknesses are, for example, from 10 nm to 5 μm for semiconductors, from 50 nm to 10 μm for the dielectric; the electrodes may, for example, be from 20 nm to 1 μm thick.

In a specific embodiment, an insulator layer (gate insulating layer) is present on at least part of the substrate surface. The insulator layer comprises at least one insulator which is preferably selected from inorganic insulators such as SiO₂, Si₃N₄, etc., ferroelectric insulators such as Al₂O₃, Ta₂O₅, La₂O₅, TiO₂, Y₂O₃, etc., organic insulators such as polyimides, benzocyclobutene (BCB), polyvinyl alcohols, polyacrylates, etc., and combinations thereof.

Suitable materials for source and drain electrodes are in principle electrically conductive materials. These include metals, preferably metals of groups 6, 8, 9, 10 or 11 of the Periodic Table, such as Pd, Au, Ag, Cu, Al, Ni, Cr, etc. Also suitable are conductive polymers such as PEDOT (=poly(3,4-ethylenedioxythiophene)):PSS (=poly(styrenesulfonate)), polyaniline, surface-modified gold, etc. Preferred electrically conductive materials have a specific resistance of less than 10⁻³ ohm×meter, preferably less than 10⁻⁴ ohm×meter, especially less than 10⁻⁶ or 10⁻⁷ ohm×meter. In a specific embodiment, drain and source electrodes are present at least partly on the organic semiconductor material. It will be appreciated that the substrate may comprise further components as used customarily in semiconductor materials or ICs, such as insulators, resistors, capacitors, conductor tracks, etc.

The electrodes may be applied by customary processes, such as evaporation, lithographic processes or another structuring process.

The compounds of the formula (I) used in accordance with the invention and the coated substrates produced therefrom are particularly advantageously suitable for use in organic field-effect transistors (OFETs). They may be used, for example, for the production of integrated circuits (ICs), for which customary n-channel MOSFETs (metal oxide semiconductor field-effect transistors) have been used to date. These are then CMOS-like semiconductor units, for example for microprocessors, microcontrollers, static RAM and other digital logic circuits. They are especially suitable for use in displays (specifically large-surface area and/or flexible displays) and RFID tags.

The compounds of the formula (I) used in accordance with the invention and the coated substrates produced therefrom are particularly advantageously suitable for use as electron conductors in organic field-effect transistors (OFETs), organic solar cells and in organic light-emitting diodes. They are also particularly advantageously suitable as exciton transport materials in excitonic solar cells.

In a preferred embodiment, the inventive field-effect transistors are thin-film transistors (TFTs). In a customary construction, a thin-film transistor has a gate electrode disposed on the substrate, a gate insulator layer disposed thereon and on the substrate, a semiconductor layer disposed on the gate insulator layer, an ohmic contact layer on the semiconductor layer, and a source electrode and a drain electrode on the ohmic contact layer.

Various semiconductor architectures based on the inventive coated substrates are also conceivable, for example top contact, top gate, bottom contact, bottom gate, or else a vertical construction, for example a VOFET (vertical organic field-effect transistor), as described, for example, in US 2004/0046182.

A further aspect of the invention relates to the provision of electronic components which are based on the inventive substrates and comprise a plurality of semiconductor components, which may be n- and/or p-semiconductors. Examples of such components are field-effect transistors (FETs), bipolar junction transistors (BJTs), tunnel diodes, converters, light-emitting components, biological and chemical detectors or sensors, temperature-dependent detectors, photodetectors such as polarization-sensitive photodetectors, gates, AND, NAND, NOT, OR, TOR and NOR gates, registers, switches, timer units, static or dynamic stores and other dynamic or sequential, logical or other digital components including programmable circuits.

By virtue of the above-described process according to the invention, it is surprisingly also possible to use the compounds of the formula (I) which are sparingly soluble per se, especially the compounds of the formula (I) in which n is from 3 to 8, in a wet processing method for producing semiconductor substrates. The compounds of the formula (I) are thus also made available for the production of semiconductor elements, especially OFETs or based on OFETs, by a printing process. In addition, the compound of the formula (I) prepared by the process according to the invention has a very high purity.

To produce OFETs, in a preferred embodiment of the process according to the invention, the surface of the substrate may be subjected to a modification before the deposition of at least one compound of the general formula (II) (and if appropriate of at least one further semiconductor material). This modification serves to form regions which bind the semiconductor materials and/or regions onto which no semiconductor materials can be deposited. Such processes are described, for example, in U.S. Ser. No. 11/353,934 (=US 2007/0190783).

A specific semiconductor element is an inverter. In digital logic, the inverter is a gate which inverts an input signal. The inverter is also referred to as a NOT gate. Real inverter circuits have an output current which constitutes the opposite of the input current. Typical values are, for example, (0, +5V) for TTL circuits. The performance of a digital inverter reproduces the voltage transfer curve (VTC), i.e. the plot of input current against output current. Ideally, it is a staged function, and the closer the real measured curve approximates to such a stage, the better the inverter is. In a specific embodiment of the invention, the compounds of the formula (I) are used as organic n-semiconductors in an inverter.

The inventive substrates coated with compounds of the formula (I) are also particularly advantageously suitable for use in organic photovoltaics (OPVs). Preference is given to the use of the inventive coated substrates in solar cells which are characterized by diffusion of excited states (exciton diffusion). In this case, one or both of the semiconductor materials utilized is notable for a diffusion of excited states (exciton mobility). Also suitable is the combination of at least one semiconductor material which is characterized by diffusion of excited states with polymers which permit conduction of the excited states along the polymer chain. In the context of the invention, such solar cells are referred to as excitonic solar cells. The direct conversion of solar energy to electrical energy in solar cells is based on the internal photo effect of a semiconductor material, i.e. the generation of electron-hole pairs by absorption of photons and the separation of the negative and positive charge carriers at a p-n transition or a Schottky contact. An exciton can form, for example, when a photon penetrates into a semiconductor and excites an electron to transfer from the valence band into the conduction band. In order to generate current, the excited state generated by the absorbed photons must, however, reach a p-n transition in order to generate a hole and an electron which then flow to the anode and cathode. The photovoltage thus generated can bring about a photocurrent in an external circuit, through which the solar cell delivers its power. The semiconductor can absorb only those photons which have an energy which is greater than its band gap. The size of the semiconductor band gap thus determines the proportion of sunlight which can be converted to electrical energy. Solar cells consist normally of two absorbing materials with different band gaps in order to very effectively utilize the solar energy. Most organic semiconductors have exciton diffusion lengths of up to 10 nm. There is still a need here for the provision of organic semiconductors through which the excited state can be passed on over very large distances. It has now been found that, surprisingly, by virtue of the process according to the invention, it is possible to provide hitherto difficult-to-obtain or unobtainable substrates coated with compounds of the formula (I), which are particularly advantageously suitable for use in excitonic solar cells.

Organic solar cells generally have a layer structure and generally comprise at least the following layers: anode, photoactive layer and cathode. These layers generally consist of a substrate customary therefor. The structure of organic solar cells is described, for example, in US 2005/0098726 and US 2005/0224905, which are fully incorporated here by reference.

Suitable substrates for this purpose are, for example, oxidic materials (such as glass, ceramic, SiO₂, especially quartz, etc.), polymers (e.g. polyvinyl chloride, polyolefins such as polyethylene and polypropylene, polyesters, fluoropolymers, polyamides, polyurethanes, polyalkyl (meth)acrylates, polystyrene and mixtures and composites thereof) and combinations thereof.

Suitable electrodes (cathode, anode) are in principle metals (preferably of groups 8, 9, 10 or 11 of the Periodic Table, e.g. Pt, Au, Ag, Cu, Al, In, Mg, Ca), semiconductors (e.g. doped Si, doped Ge, indium tin oxide (ITO), gallium indium tin oxide (GITO), zinc indium tin oxide (ZITO), etc.), metal alloys (e.g. based on Pt, Au, Ag, Cu, etc., especially Mg/Ag alloys), semiconductor alloys, etc. The anode used is preferably a material essentially transparent to incident light. This includes, for example, ITO, doped ITO, ZnO, TiO₂, Ag, Au, Pt. The cathode used is preferably a material which essentially reflects the incident light. This includes, for example, metal films, for example of Al, Ag, Au, In, Mg, Mg/Al, Ca, etc.

For its part, the photoactive layer comprises at least one or consists of at least one layer which has been produced by a process according to the invention and which comprises, as an organic semiconductor material, at least one compound of the formula (I) as defined above. In a specific embodiment, the photoactive layer comprises at least one organic acceptor material. In addition to the photoactive layer, there may be one or more further layers, for example a layer with electron-conducting properties (ETL, electron transport layer) and a layer which comprises a hole-conducting material (hole transport layer, HTL) which need not absorb, exciton- and hole-blocking layers (e.g. EBLs) which should not absorb, multiplication layers. Suitable exciton- and hole-blocking layers are described, for example, in U.S. Pat. No. 6,451,415.

Suitable exciton blocker layers are, for example, bathocuproins (BCPs), 4,4′,4″-tris[3-methylphenyl-N-phenylamino]triphenylamine (m-MTDATA) or polyethylenedioxy-thiophene (PEDOT), as described in U.S. Pat. No. 7,026,041.

The inventive excitonic solar cells are based on photoactive donor-acceptor heterojunctions. When at least one compound of the formula (I) is used as the HTM (hole transport material), the corresponding ETM (exciton transport material) must be selected such that, after excitation of the compounds, a rapid electron transfer to the ETM takes place. Suitable ETMs are, for example, C60 and other fullerenes, perylene-3,4:9,10-bis(dicarboximides) (PTCDIs), etc. When at least one compound of the formula (I) is used as the ETM, the complementary HTM must be selected such that, after excitation of the compound, a rapid hole transfer to the HTM takes place. The heterojunction may have a flat configuration (cf. Two layer organic photovoltaic cell, C. W. Tang, Appl. Phys. Lett., 48 (2), 183-185 (1986) or N. Karl, A. Bauer, J. Holzäpfel, J. Marktanner, M. Möbus, F. Stölzle, Mol. Cryst. Liq. Cryst., 252, 243-258 (1994).) or be implemented as a bulk heterojunction (or interpenetrating donor-acceptor network; cf., for example, C. J. Brabec, N. S. Sariciftci, J. C. Hummelen, Adv. Funct. Mater., 11 (1), (2001).).

The photoactive layer based on a heterojunction between at least one compound of the formula (I) and an HTL (hole transport layer) or ETL (exciton transport layer) can be used in solar cells with MiM, pin, pn, Mip or Min structure (M=metal, p=p-doped organic or inorganic semiconductor, n=n-doped organic or inorganic semiconductor, i=intrinsically conductive system of organic layers; cf., for example, J. Drechsel et al., Org. Eletron., 5 (4), 175 (2004) or Maennig et al., Appl. Phys. A 79, 1-14 (2004)). This layer can also be used in tandem cells, as described by P. Peumans, A. Yakimov, S. R. Forrest in J. Appl. Phys, 93 (7), 3693-3723 (2003) (cf. patents U.S. Pat. No. 4,461,922, U.S. Pat. No. 6,198,091 and U.S. Pat. No. 6,198,092). It can also be used in tandem cells composed of two or more MiM, pin, Mip or Min diodes stacked on one another (cf. patent application DE 103 13 232.5) (J. Drechsel et al., Thin Solid Films, 451452, 515-517 (2004)).

The layer thicknesses of the M, n, i and p layers are typically from 10 to 1000 nm, preferably from 10 to 400 nm. Thin layers can be produced by vapor deposition under reduced pressure or in inert gas atmosphere, by laser ablation or by solution- or dispersion-processible methods such as spin-coating, knife-coating, casting methods, spraying, dip-coating or printing (e.g. inkjet, flexographic, offset, gravure; intaglio, nanoimprinting).

In addition to the compounds of the general formula (I), the following semiconductor materials are suitable for use in organic photovoltaics:

Phthalocyanines, for example phthalocyanines which bear at least one halogen substituent, such as hexadecachlorophthalocyanines and hexadecafluorophthalocyanines, metal-free phthalocyanines or phthalocyanines comprising divalent metals or metal atom-containing groups, especially those of titanyloxy, vanadyloxy, iron, copper, zinc, etc. Suitable phthalocyanines are especially copper phthalocyanine, zinc phthalocyanine, metal-free phthalocyanine, copper hexadecachlorophthalocyanine, zinc hexadecachlorophthalocyanine, metal-free hexadecachlorophthalocyanine, copper hexadecafluorophthalocyanine, hexadecafluorophthalocyanine or metal-free hexadecafluorophthalocyanine;

porphyrins, for example 5, 10,15,20-tetra(3-pyridyl)porphyrin (TpyP), or else tetrabenzoporphyrins, for example metal-free tetrabenzoporphyrin, copper tetrabenzoporphyrin or zinc tetrabenzoporphyrin. Especially preferred are tetrabenzoporphyrins which, like the compounds of the formula (I) used in accordance with the invention, are processed from solution as soluble precursors and are converted to the pigmentary photoactive component by thermolysis on the substrate; liquid-crystalline (LC) materials, for example coronenes, such as hexabenzocoronene (HBC-PhC₁₂), coronenediimides, or triphenylenes such as 2,3,6,7,10,11-hexahexylthiotriphenylene (HTT₆), 2,3,6,7,10,11-hexakis(4-n-nonylphenyl)triphenylene (PTP₉) or 2,3,6,7,10,11-hexakis(undecyloxy)triphenylene (HAT₁₁). Particular preference is given to liquid-crystalline materials which are discotic;

Thiophenes, oligothiophenes and substituted derivatives thereof. Suitable oligothiophenes are quaterthiophenes, quinquethiophenes, sexithiophenes, α,ω-di(C₁-C₈)alkyloligothiophenes such as α,ω-dihexylquaterthiophenes, α,ω-dihexylquinquethiophenes and α,ω-dihexylsexithiophenes, poly(alkylthiophenes) such as poly(3-hexylthiophene), bis(dithienothiophenes), anthradithiophenes and dialkylanthradithiophenes such as dihexylanthradithiophene, phenylene-thiophene (P-T) oligomers and derivatives thereof, especially α,ω-alkyl-substituted phenylene-thiophene oligomers;

also suitable are compounds of the α,α′-bis(2,2-dicyanovinyl)quinquethiophene (DCV₅T) type, poly[3-(4-octylphenyl)-2,2′-bithiophene] (PTOPT), poly(3-(4′-(1,4,7-trioxaoctyl)phenyl)thiophene (PEOPT), poly(3-(2′-methoxy-5′-octylphenyl)thiophene) (POMeOPT), poly(3-octylthiophene) (P₃OT), poly(pyridopyrazinevinylene)-polythiophene blends such as EHH-PpyPz, PTPTB copolymers, BBL, poly(9,9-dioctylfluorene-co-bis-N,N′-(4-methoxyphenyl)bis-N,N′-phenyl-1,4-phenylenediamine) (PFMO), see Brabec C., Adv. Mater., 2996, 18, 2884, (PCPDTBT) poly[2,6-(4,4-bis(2-ethylhexyl)-4H-cyclopenta[2,1-b;3,4-b′]-dithiophene)-4,7-(2,1,3-benzothiadiazole)];

paraphenylenevinylene and paraphenylenevinylene-comprising oligomers or polymers, for example polyparaphenylenevinylene (PPV), MEH-PPV (poly(2-methoxy-5-(2′-ethylhexyloxy)-1,4-phenylenevinylene)), MDMO-PPV (poly(2-methoxy-5-(3′,7′-dimethyloctyloxy)-1,4-phenylenevinylene)), cyanoparaphenylenevinylene (CN-PPV), CN-PPV modified with various alkoxy derivatives;

phenyleneethynylene/phenylenevinylene hybrid polymers (PPE-PPV);

polyfluorenes and alternating polyfluorene copolymers, for example with 4,7-dithien-2′-yl-2,1,3-benzothiadiazole. Also suitable are poly(9,9′-dioctylfluorene-co-benzothiadiazole) (F₈BT), poly(9,9′-dioctylfluorene-co-bis(N,N′-(4-butylphenyl))-bis(N,N′-phenyl)-1,4-phenylenediamine (PFB);

polycarbazoles, i.e. carbazole-comprising oligomers and polymers;
polyanilines, i.e. aniline-comprising oligomers and polymers;
triarylamines, polytriarylamines, polycyclopentadienes, polypyrroles, polyfurans, polysiloles, polyphospholes, N,N′-bis(3-methylphenyl)-N,N′-bis(phenyl)benzidine (TPD), 4,4′-bis(carbazol-9-yl)biphenyl (CBP), 2,2′,7,7′-tetrakis(N,N-di-p-methoxyphenylamino)-9,9′-spirobifluorene (Spiro-MeOTAD);

fullerenes, especially C₆₀ and derivatives thereof such as PCBM (=[6,6]-phenyl-C₆₁-butyric acid methyl ester); in such cells, the fullerene derivative is a hole conductor.

All aforementioned p-semiconductor materials may also be doped. Suitable examples of dopants for p-semiconductors are 2,3,5,6-tetrafluoro-7,7,8,8-tetracyanoquino-dimethane (F₄-TCNQ), etc.

The invention further provides an organic light-emitting diode (OLED) which comprises at least one inventive substrate coated with compounds of the formula (I). The compounds of the formula (I) may serve as a charge transport material (electron conductor).

Organic light-emitting diodes are in principle constructed from several layers. These include 1. anode, 2. hole-transporting layer, 3. light-emitting layer, 4. electron-transporting layer and 5. cathode. It is also possible that the organic light-emitting diode does not have all of the layers mentioned; for example, an organic light-emitting diode with the layers (1) (anode), (3) (light-emitting layer) and (5) (cathode) is likewise suitable, in which case the functions of the layers (2) (hole-transporting layer) and (4) (electron-transporting layer) are assumed by the adjacent layers. OLEDs which have the layers (1), (2), (3) and (5) or the layers (1), (3), (4) and (5) are likewise suitable. The structure of organic light-emitting diodes and processes for their production are known in principle to those skilled in the art, for example from WO 2005/019373. Suitable materials for the individual layers of OLEDs are disclosed, for example, in WO 00/70655. Reference is made here to the disclosure of these documents. The inventive OLEDs are notable in that at least one layer which comprises a compound of the formula (I) is provided by solution processing at least one compound of the formula (II) and then converting these compounds to compounds of the formula (I) by heating the substrate.

Suitable substrates are, for example, glass or polymer films. The organic layers can be provided from solutions or dispersions in suitable solvents, for which coating techniques known to those skilled in the art are employed. Alternatively, the organic layers which do not comprise any compounds of the formula (I) can also be produced by vapor deposition by customary techniques, i.e. by thermal evaporation, chemical vapor deposition and others. In an alternative process can

As explained above, the compounds of the formula (II) used in accordance with the invention are notable in that a controlled conversion of these compounds to compounds of the formula (I) can be brought about actually at temperatures at which the compounds of the formula (II) are not subject to any further undefined decomposition reactions.

The present invention therefore further relates to a process for preparing compounds of the formula (I), as defined above, in which
A) a compound of the formula (II), as defined above, is provided,
B) the compound of the formula (II) is heated to a temperature at which at least some of the compound of the formula (II) is converted to a compound of the formula (I).

With regard to the heating of the compounds of the formula (II), full reference is made to the above for step ii) of the process according to the invention for producing coated substrates.

For the further purification of the compounds of the formula (I) obtained by a process according to the invention, it is possible to recrystallize, for example, from a mixture of halogenated solvents such as chloroform and methylene chloride, and alcohols such as methanol, ethanol and isopropanol. Alternatively, it is also possible to undertake column chromatography on silica gel using methylene chloride or acetone as the eluent.

A further purification method consists in recrystallizing the compounds of the formula (I) from N,N-disubstituted aliphatic carboxamides such as N,N-dimethylformamide and N,N-dimethylacetamide, or nitrogen-containing heterocycles such as N-methylpyrrolidone, or mixtures thereof with alcohols such as methanol, ethanol and isopropanol, or washing them with these solvents.

Finally, the compounds of the formula (I) can also be fractionated from sulfuric acid.

Compounds of the formula (II) in which the R^{A} and R^{B} radicals have one of the definitions given above are additionally notable in that the differently substituted rylenes which are typically obtained when they are provided can be separated in a particularly advantageous manner by chromatography, and so compounds of the formula (II) of particularly high purity can be provided.

Therefore, a specific embodiment of the invention relates to a process according to the invention for preparing compounds of the formula (I), in which the provision of the compound of the formula (II) in step A) comprises the chromatographic separation of a mixture comprising the compound of the formula (II).

The invention further relates to compounds of the formula (I)′

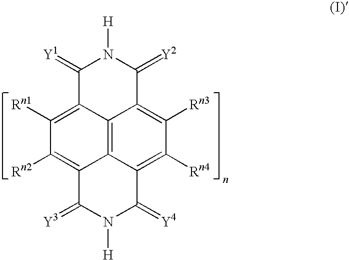

in which
n is 4,
Y¹, Y², Y³ and Y⁴ are each independently O or S and Rⁿ¹, Rⁿ², Rⁿ³ and Rⁿ⁴ are each independently hydrogen or cyano, where two of the Rⁿ¹ and Rⁿ² radicals and/or Rⁿ³ and Rⁿ⁴ radicals in each case may together also be part of an aromatic ring system fused to one or two adjacent naphthalene units of the rylene skeleton,
   where at least one of the Rⁿ¹, Rⁿ², Rⁿ³ and Rⁿ⁴ radicals is CN. These compounds are not known from the prior art and are advantageously suitable for use in emitter materials, charge transport materials or exciton transport materials. More particularly, the compounds of the formula (I)′ are suitable for coating substrates in the process according to the invention.

Preferably, in the compounds of the formula (I)′, Y¹, Y², Y³ and Y⁴ are each O.

Likewise preferably, in the compounds of the formulae (I)′, from 1 to (2n−2) of the Rⁿ¹, Rⁿ², Rⁿ³ and Rⁿ⁴ radicals are CN, i.e., in the case of terrylenes (n=3), from 1 to 4, preferably 2 or 4, of the Rⁿ¹, Rⁿ², Rⁿ³ and Rⁿ⁴ radicals are CN and, in the case of quaterrylenes (n=4), from 1 to 6, preferably 2, 4 or 6.

The invention further relates to compounds of the formula (II)′

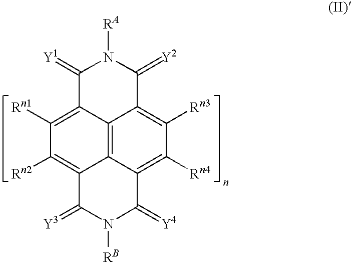

in which
n is 3 or 4,
Y¹, Y², Y³ and Y⁴ are each independently O or S,
Rⁿ¹, Rⁿ², Rⁿ³ and Rⁿ⁴ are each independently hydrogen or cyano, where two of the Rⁿ¹ and Rⁿ² radicals and/or Rⁿ³ and Rⁿ⁴ radicals in each case may together also be part of an aromatic ring system fused to one or two adjacent naphthalene units of the rylene skeleton,
   where at least one of the Rⁿ¹, Rⁿ², Rⁿ³ and Rⁿ⁴ radicals is CN,
R^{A} and R^{B} are each independently a group of the formula (III)

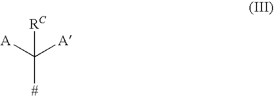

in which
# in each case represents the bond to the nitrogen atom,
A and A′ are each independently in each case optionally substituted C₁-C₂₅-alkyl, C₂-C₂₅-alkenyl, C₂-C₂₅-alkynyl, aryl or hetaryl, where C₁-C₂₅-alkyl, C₂-C₂₅-alkenyl and C₂-C₂₅-alkynyl may each be interrupted once or more than once by O, S, NR^{a}, —C(═O)—, —C(═O)O—, —C(═O)N(R^{a})—, —S(═O)₂O— or —S(═O)₂N(R^{a})—, in which
   R^{a} is selected from in each case optionally substituted C₁-C₁₂-alkyl, aryl and hetaryl, and
R^{C} is hydrogen or in each case optionally substituted C₁-C₁₂-alkyl, C₂-C₁₂-alkenyl, C₂-C₁₂-alkynyl, aryl or hetaryl, where C₁-C₂₅-alkyl, C₂-C₂₅-alkenyl and C₂-C₂₅-alkynyl may each be interrupted once or more than once by O, S, NR^{a}, —C(═O)—, —C(═O)O—, —C(═O)N(R^{a})—, —S(═O)₂O— or —S(═O)₂N(R^{a})—.

The compounds of the formule (I)′ and (II)′ are not described in the prior art and are advantageously suitable for use in the processes according to the invention, for the coating of substrates or for the preparation of the compounds of the formula (I).

The present invention therefore further relates to the use of a solution of compounds of the formula (II)′ for treatment of substrates, wherein the substrates are coated over at least part of their surface area with compounds of the formula (II)′ by the treatment.

In the compounds of the formula (II)′, preferably at least one of the A and A′ radicals in the groups of the formula (III) is unsubstituted or substituted C₁-C₂₅-alkyl, unsubstituted or substituted C₃-C₂₅-alkenyl or unsubstituted or substituted C₃-C₂₅-alkynyl with at least one hydrogen atom in the beta position to the nitrogen atom of the rylene skeleton, and where C₁-C₂₅-alkyl, C₃-C₂₅-alkenyl and C₃-C₂₅-alkynyl may each be interrupted once or more than once, for example once, twice, thrice or more than thrice, by O, S, NR^{a}, —C(═O)—, —C(═O)O—, —C(═O)N(R^{a})—, —S(═O)₂O— or —S(═O)₂N(R^{a})—, and R^{a} is as defined above.

In the compounds of the formula (II)′, preferably from 1 to (2n−2) of the Rⁿ¹, Rⁿ², Rⁿ³ and Rⁿ⁴ radicals is CN, i.e., in the case of terrylenes (n=3), from 1 to 4, preferably 2 or 4, of the Rⁿ¹, Rⁿ², Rⁿ³ and Rⁿ⁴ radicals are CN and, in the case of quaterrylenes (n=4), from 1 to 6, preferably 2, 4 or 6.

With regard to preferred definitions of the Rⁿ¹, Rⁿ², Rⁿ³, Rⁿ⁴, R^{A} and R^{B} radicals, full reference is made to the remarks made regarding the compounds of the formula (II).

Preferably, in the compounds of the formula (II)′, Y¹, Y², Y³ and Y⁴ are each O.

Likewise preferably, in the compounds of the formula (II)′, n is 4.

In the compounds of the formula (II)′, at least one of the A and A′ radicals is, and especially both A and A′ radicals in the groups of the formula (III) are, preferably C₄-C₂₅-alkyl. Preferably, at least one of the A and A′ radicals has a hydrogen atom in the beta position to the nitrogen atom of the rylene skeleton.

In the compounds of the formula (II)′, R^{C} in the groups of the formula (III) is preferably hydrogen.

The compounds of the formula (I)′ or (II)′ in which 1 or 2 of the Rⁿ¹, Rⁿ², Rⁿ³ and Rⁿ⁴ radicals are CN can be prepared proceeding from compounds which have the same rylene base skeleton and possess 1 or 2 exchangeable bromine or chlorine atoms as Rⁿ¹, Rⁿ², Rⁿ³ and Rⁿ⁴ radicals, through exchange of the bromine or chlorine atoms for cyano. The conditions for such an exchange reaction are known per se to those skilled in the art.

For the exchange of bromine or chlorine for cyano, suitable are mono- or divalent metal cyanides, for example alkali metal cyanides such as KCN and NaCN, or zinc cyanide. The reaction is effected preferably in the presence of at least one transition metal catalyst. Suitable transition metal catalysts are especially palladium complexes such as tetrakis(triphenylphosphine)palladium(0), tetrakis(tris-o-tolylphosphine)palladium(0), [1,2-bis(diphenylphosphino)ethane]palladium(II) chloride, [1,1′-bis(diphenylphosphino)ferrocene]palladium(II) chloride, bis(triethylphosphine)-palladium(II) chloride, bis(tricyclohexylphosphine)palladium(II) acetate, (2,2′-bipyridyl)palladium(II) chloride, bis(triphenylphosphine)palladium(II) chloride, tris(dibenzylideneacetone)dipalladium(0), 1,5-cyclooctadienepalladium(II) chloride, bis(acetonitrile)palladium(II) chloride and bis(benzonitrile)palladium(II) chloride, preference being given to [1,1′-bis(diphenylphosphino)ferrocene]palladium(II) chloride and tetrakis(triphenylphosphine)palladium(0).

For the exchange of bromine or chlorine for cyano, preference is given to using aromatic hydrocarbons as solvents. These preferably include benzene, toluene, xylenes, etc. Particular preference is given to using toluene.

The present invention further relates to compounds of the formula (II)″

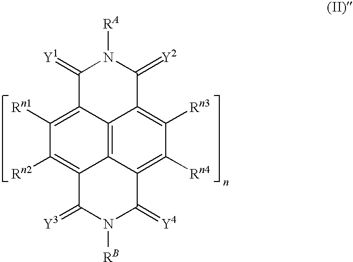

in which
n is an integer from 1 to 8
Y¹, Y², Y³ and Y⁴ are each independently O or S and
Rⁿ¹, Rⁿ², Rⁿ³ and Rⁿ⁴ are each independently hydrogen, F, Cl, Br, CN, alkoxy, alkylthio, alkylamino, dialkylamino, aryloxy, arylthio, hetaryloxy or hetarylthio, where two of the Rⁿ¹ and Rⁿ² radicals and/or Rⁿ³ and Rⁿ⁴ radicals in each case may together also be part of an aromatic ring system fused to one or two adjacent naphthalene units of the rylene skeleton, and
R^{A} and R^{B} are each independently a group of the formula (III)′

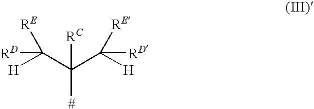

in which
# in each case represents the bond to the nitrogen atom,
R^{C} is hydrogen or C₁-C₁₂-alkyl and
R^{D}, R^{D′}, R^{E} and R^{E′} are each independently C₁-C₁₂-alkyl.

With the exception of N,N′-bis(1-isopropyl-2-methylpropyl)perylene-3,4:9,10-tetra-carboximide, N,N′-bis[2-ethyl-1-(1-ethylpropyl)butyl]perylene-3,4:9,10-tetracarboximide and N,N′-bis[2-propyl-1-(1-propylbutyl)pentyl]perylene-3,4:9,10-tetracarboximide, which have been described as compounds with pronounced solid-state fluorescence (see DE 10 2004 024 909 A1; Angew. Chem. 2005, 117, 2479-2480), the compounds of the formula (II)″ are not known from the prior art and are advantageously suitable for use in the process according to the invention for coating substrates or for preparing compounds of the formula (I).

The present invention therefore further relates to the use of a solution of compounds of the formula (II)″ for treatment of substrates, wherein the substrates are coated over at least part of their surface area with compounds of the formula (II)″ by the treatment.

Especially advantageous with regard to the processes according to the invention is the use of compounds of the formula (II)″ in which n is an integer from 3 to 8, and especially the use of compounds of the formula (II)″ in which n is 3 or 4. Accordingly, preference is given to those compounds of the formula (II)″.

With regard to preferred embodiments of the Y¹, Y², Y³, Y⁴, Rⁿ¹, Rⁿ², Rⁿ³, Rⁿ⁴, R^{C}, R^{D}, R^{D′}, R^{E} and R^{E′} radicals, full reference is made to the remarks made regarding the compounds of the formula (II).

In a specific embodiment of the present invention, in the compounds of the formula (II)″, R^{c} in the group of the formula (III) is hydrogen. In a further specific embodiment, in the compounds of the formula (II)″, R^{C} in the group of the formula (III) is C₁-C₁₂-alkyl.

The present invention further relates to compounds of the formula (II)′″

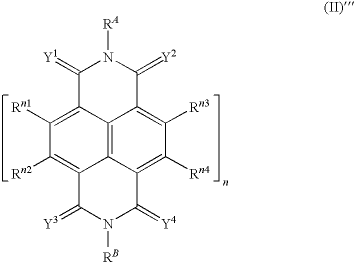

in which
n is an integer from 5 to 8
Y¹, Y², Y³ and Y⁴ are each independently O or S and
Rⁿ¹, Rⁿ², Rⁿ³ and Rⁿ⁴ are each independently hydrogen, F, Cl, Br, CN, alkoxy, alkylthio, alkylamino, dialkylamino, aryloxy, arylthio, hetaryloxy or hetarylthio, where two of the Rⁿ¹ and Rⁿ² radicals and/or Rⁿ³ and Rⁿ⁴ radicals in each case may together also be part of an aromatic ring system fused to one or two adjacent naphthalene units of the rylene skeleton, and
R^{A} and R^{B} are each independently a group of the formula (III)

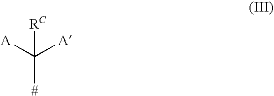

in which
# in each case represents the bond to the nitrogen atom,
A and A′ are each independently in each case optionally substituted C₁-C₂₅-alkyl, C₂-C₂₅-alkenyl, C₂-C₂₅-alkynyl, aryl or hetaryl, where C₁-C₂₅-alkyl, C₂-C₂₅-alkenyl and C₂-C₂₅-alkynyl may each be interrupted once or more than once by O, S, NR^{a}, —C(═O)—, —C(═O)O—, —C(═O)N(R^{a})—, —S(═O)₂O— or —S(═O)₂N(R^{a})—, in which
   R^{a} is selected from in each case optionally substituted C₁-C₁₂-alkyl, aryl or hetaryl, and
R^{C} is hydrogen or in each case optionally substituted C₁-C₁₂-alkyl, C₂-C₁₂-alkenyl, C₂-C₁₂-alkynyl, aryl or hetaryl, where C₁-C₂₅-alkyl, C₂-C₂₅-alkenyl and C₂-C₂₅-alkynyl may each be interrupted once or more than once by O, S, NR^{a}, —C(═O)—, —C(═O)O—, —C(═O)N(R^{a})—, —S(═O)₂O— or —S(═O)₂N(R^{a})—.

With the exception of N,N′-bis(1-heptyloctyl)pentarylene-3,4:15,16-tetracarboximide (see DE 10 2005 018 241; Angew. Chem. Int. Ed. 2006, 45, 1401-1404), the compounds of the formula (II)′″ are not known from the prior art and are advantageously suitable for use in the process according to the invention, for the coating of substrates or for the preparation of compounds of the formula (I).

The present invention therefore further relates to the use of a solution of compounds of the formula (II)′″ for treatment of substrates, wherein the substrates are coated over at least part of their surface area with compounds of the formula (II)′″ by the treatment.

With regard to preferred definitions of the Y¹, Y², Y³, Y⁴, Rⁿ¹, Rⁿ², Rⁿ³, Rⁿ⁴, R^{A} and R^{B} radicals, full reference is made to the remarks made regarding the compounds of the formula (II).

In the groups of the formula (III), preferably at least one of the A and A′ radicals in the compounds of the formula (III)′″ is unsubstituted or substituted C₁-C₂₅-alkyl, unsubstituted or substituted C₃-C₂₅-alkenyl or unsubstituted or substituted C₃-C₂₅-alkynyl, the three radicals mentioned having at least one hydrogen atom in the beta position to the nitrogen atom of the rylene skeleton, and where C₁-C₂₅-alkyl, C₃-C₂₅-alkenyl and C₃-C₂₅-alkynyl may each be interrupted once or more than once, for example once, twice, thrice or more than thrice, by O, S, NR^{a}, —C(═O)—, —C(═O)O—, —C(═O)N(R^{a})—, —S(═O)₂O— or —S(═O)₂N(R^{a})—, and R^{a} is as defined above.

In addition, in a preferred embodiment, in the compounds of the formula (II)′″, at least one of the A and A′ radicals in the groups of the formula (III) is, and especially both A and A′ radicals are, each C₄-C₂₅-alkyl. Preferably at least one of the A and A′ radicals has a hydrogen atom in the beta position to the nitrogen atom of the rylene skeleton.

In a specific embodiment of the present invention, in the compounds of the formula (II)′″, R^{c} in the group of the formula (III) is hydrogen. In a further specific embodiment, R^{C} is C₁-C₁₂-alkyl.

### DESCRIPTION OF THE FIGURES

FIG. 1 shows the analysis results of the thermogravimetry analysis (TGA) as a function of temperature for the decomposition of N,N′-bis(1-heptyloctyl)quaterrylene-3,4:13,14-tetracarboximide, at a temperature gradient of 1° C./min and a maximum temperature of 500° C.

FIG. 2 shows the analysis results of the thermogravimetry analysis (TGA) as a function of time for the decomposition of N,N′-bis(1-heptyloctyl)quaterrylene-3,4:13,14-tetracarboximide, at a temperature gradient of 1° C./min, a minimum temperature of 30° C. and a maximum temperature of 420° C., at which the sample studied was held for a further 10 minutes.

FIG. 3 shows the analysis results of the thermogravimetry analysis (TGA) as a function of time for the decomposition of N,N′-bis(1-heptyloctyl)quaterrylene-3,4:13,14-tetracarboximide, at a temperature gradient of 10° C./min, a minimum temperature of 30° C. and a maximum temperature of 405° C., at which the sample studied was held for a further 10 minutes.

FIG. 4 shows the analysis results of the thermogravimetry analysis (TGA) as a function of temperature for the decomposition of N,N′-bis(1-hexylheptyl)perylene-3,4:13,14-tetracarboximide, at a temperature gradient of 10° C./min and a maximum temperature of 500° C.

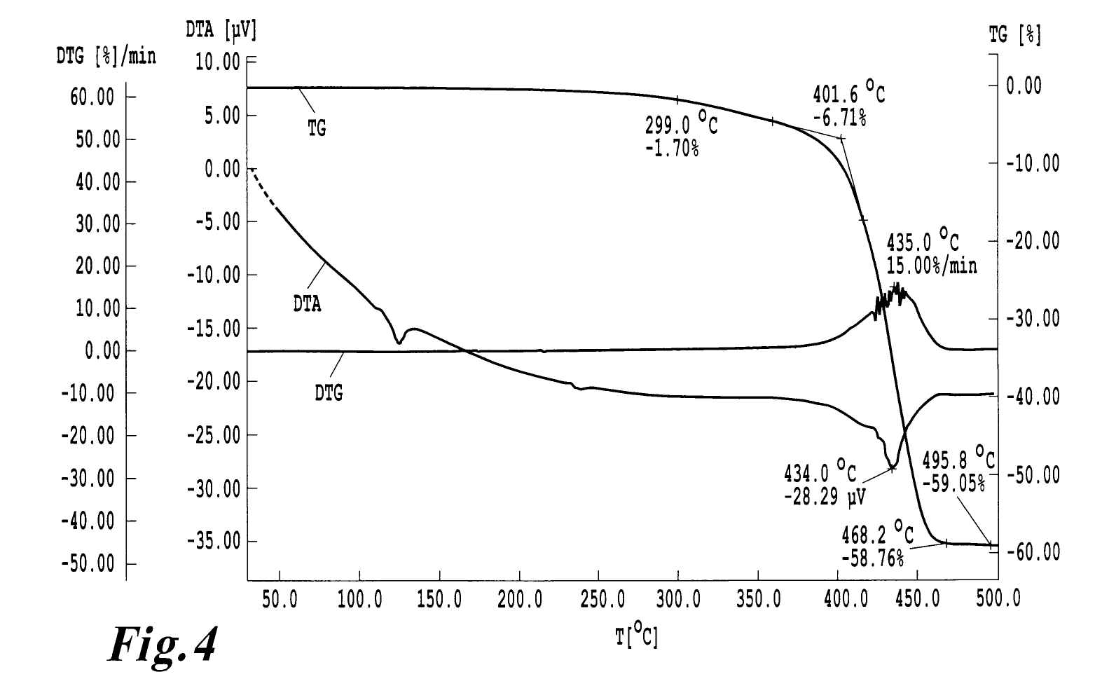

FIG. 5 (noninventive) shows the analysis results of the thermogravimetry analysis (TGA) as a function of temperature for the decomposition of N,N′-bis(methyl)-quaterrylene-3,4:13,14-tetracarboximide, at a temperature gradient of 10° C./min and a maximum temperature of 700° C.

FIG. 6 shows the analysis results of the thermogravimetry analysis (TGA) as a function of temperature for the decomposition of N,N′-bis(4,6-dipropylnon-5-yl)quaterrylene-3,4:13,14-tetracarboximide at a temperature gradient of 10° C./min and a maximum temperature of 600° C.

FIG. 7 shows the analysis results of the thermogravimetry analysis (TGA) as a function of temperature for the decomposition of N,N′-bis(1-ethylbenzyl)perylene-3,4:9,10-tetracarboximide at a temperature gradient of 10° C./min and a maximum temperature of 450° C.

### EXAMPLES

The invention will be illustrated further hereinafter with reference to nonlimiting examples.

### I) Examples of the Thermal Decomposition of Compounds of the Formula (I)

To determine the thermal properties of the rylene compounds, the TG/DTA (thermogravimetry/differential thermoanalysis) system from Seiko was used.

The rylene compound is weighed into a platinum crucible. The reference used is aluminum oxide (23.20 mg). The thermogravimetry analysis is carried out under a nitrogen atmosphere.

### Example I.1

### Decomposition of N,N′-bis(1-heptyloctyl)quaterrylene-3,4:13,14-tetracarboximide

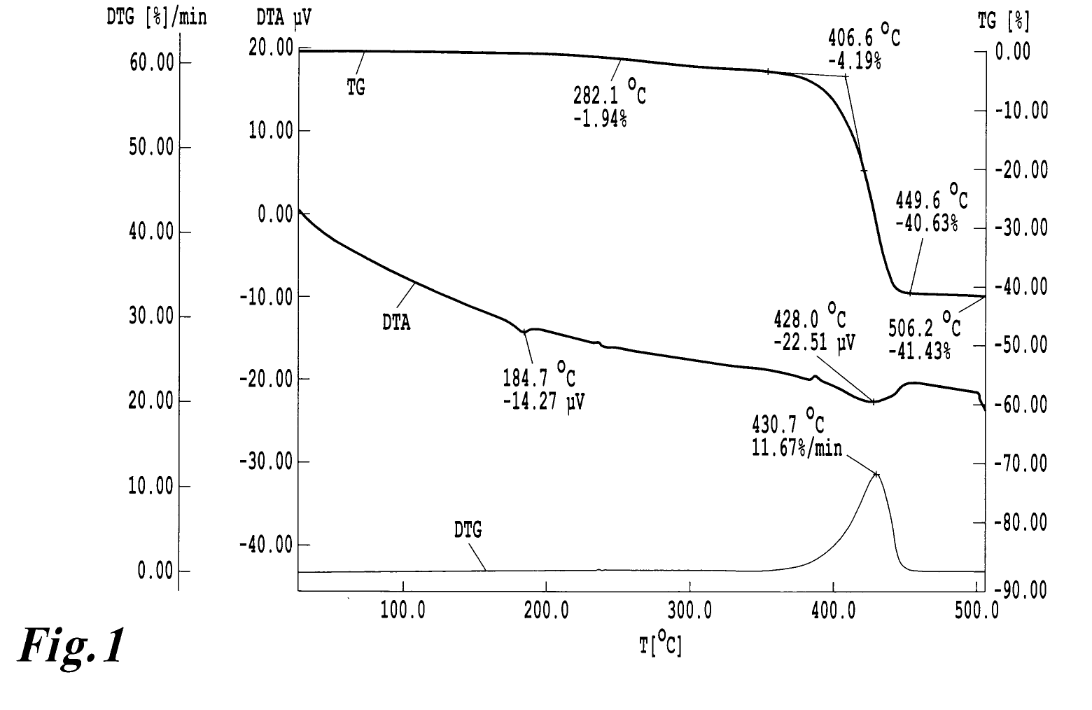

a) A sample of N,N′-bis(1-heptyloctyl)quaterrylene-3,4:13,14-tetracarboximide (12.56 mg) was weighed and analyzed by thermogravimetry. The temperature was increased with a gradient of 10° C./min from 30° C. to 500° C. The results as a function of temperature are reproduced in FIG. 1. A defined decrease in the weight of the sample was observed within a temperature range of from 406 to 450° C. with a maximum of 11.7%/min at a temperature of 431° C. The weight of the sample decreased by 41.4%. This corresponds approximately to the proportion by weight of the two 1-heptyloctyl groups based on the total weight of the compound used. No further decomposition was observed within the analysis range.

The crucible residue of the thermogravimetric was analyzed by UV spectroscopy and mass spectroscopy. The molar extinction of a sample of the crucible residue in H₂SO₄ (conc.) at a wavelength of 869 nm was 619 500 l/mol·cm. This corresponds to a very high purity of the corresponding N,N′-unsubstituted compound. MALDI-MS: 638.1 g/mol.

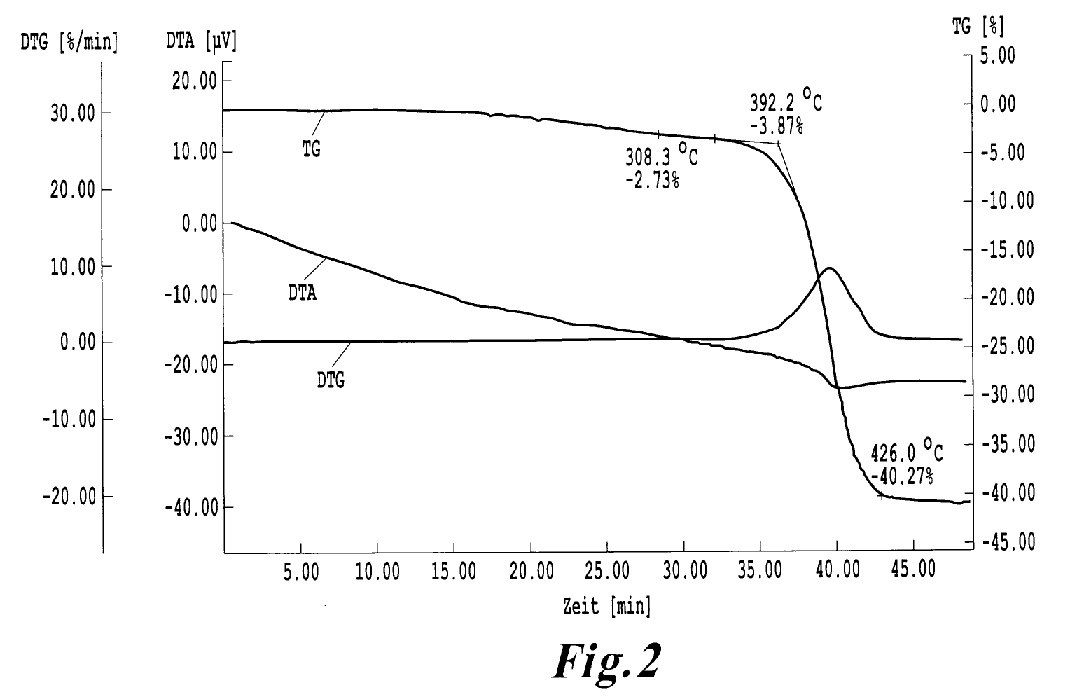

b) A sample of N,N′-bis(1-heptyloctyl)quaterrylene-3,4:13,14-tetracarboximide (8.71 mg) was weighed and analyzed by thermogravimetry. The temperature was increased with a gradient of 10° C./min from 30° C. to 420° C. and held at this temperature for a further 10 minutes. The results as a function of time are reproduced in FIG. 2. A defined decrease in the weight of the sample was observed within a range from 33 to 43 with a maximum of about 10%/min at 39 minutes. The weight of the sample decreased by 40.3%. This corresponds approximately to the proportion by weight of the two 1-heptyloctyl groups based on the total weight of the compound used. No further decomposition was observed within the analysis range. The molar extinction of a sample of the crucible residue in H₂SO₄ (conc.) at a wavelength of 869 nm was 647 382 l/mol·cm.

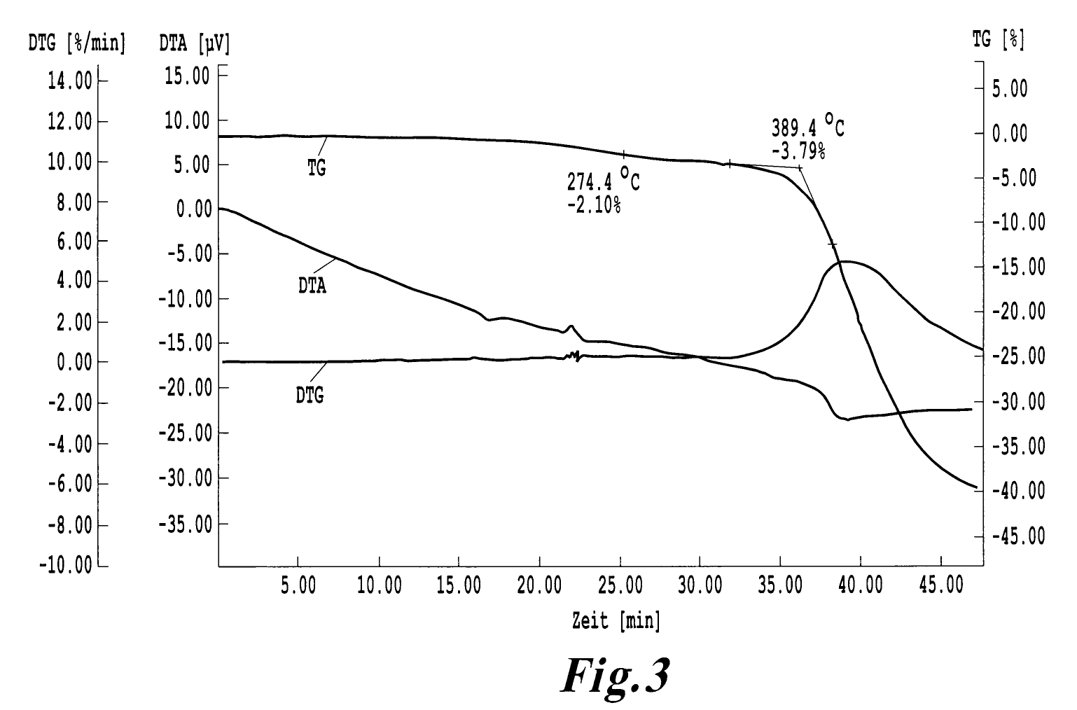

c) A sample of N,N′-bis(1-heptyloctyl)quaterrylene-3,4:13,14-tetracarboximide (9.88 mg) was weighed and analyzed by thermogravimetry. The temperature was increased with a gradient of 10° C./min from 30° C. to 405° C. and held at this temperature for a further 10 minutes. The results as a function of time are reproduced in FIG. 3. A significant decrease in the weight of the sample was observed within a range from 33 to 47 minutes with a maximum of about 10%/min at 39 minutes. The weight of the sample decreased by 40.0%. This corresponds approximately to the proportion by weight of the two 1-heptyloctyl groups based on the total weight of the compound used. No further decomposition was observed within the analysis range. The molar extinction of a sample of the crucible residue in H₂SO₄ (conc.) at a wavelength of 869 nm was 638 722 l/mol·cm.

### Example I.2

### Decomposition of N,N′-bis(1-hexylheptyl)perylene-3,4:9,10-tetracarboximide

A sample of N,N′-bis(1-hexylheptyl)perylene-3,4:9,10-tetracarboximide (16.71 mg) was weighed and analyzed by thermogravimetry. The temperature was increased with a gradient of 10° C./min from 30° C. to 500° C. The results as a function of temperature are reproduced in FIG. 1. A defined decrease in the weight of the sample was observed within a temperature range of from 360 to 496° C. with a maximum of 15.0%/min at a temperature of 435° C. The weight of the sample decreased by 59.1%. This corresponds approximately to the proportion by weight of the two 1-hexylheptyl groups based on the total weight of the compound used. No further decomposition was observed within the analysis range.

The crucible residue of the thermogravimetric was analyzed by mass spectroscopy. Apart from the mass corresponding to perylene-3,4:9,10-tetracarboximide, no further masses of decomposition products were detected.

### Example I.3 (comparative example)

### Decomposition of N,N′-bis(methyl)perylene-3,4:9,10-tetracarboximide

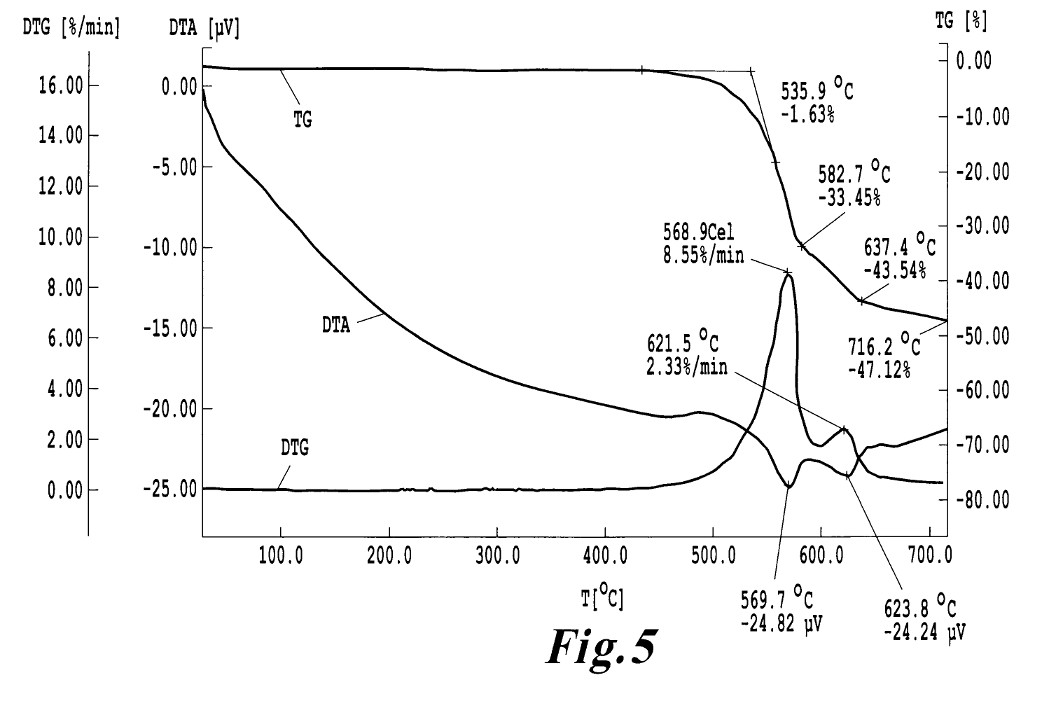

A sample of N,N′-bis(methyl)perylene-3,4:9,10-tetracarboximide (15.60 mg) was weighed and analyzed by thermogravimetry. The temperature was increased with a gradient of 10° C./min from 30° C. to 700° C. The results as a function of temperature are reproduced in FIG. 5. From a temperature of 500° C., a significant decrease in the weight of the sample was observed, which had not ended even on attainment of the maximum temperature of 700° C. No decrease in the weight of the sample which can be attributed to the defined elimination of the two N-bonded methyl groups was observed.

### Example I.4

### Decomposition of N,N′-bis(4,6-dipropylnon-5-yl)quaterrylene-3,4:13,14-tetracarboximide

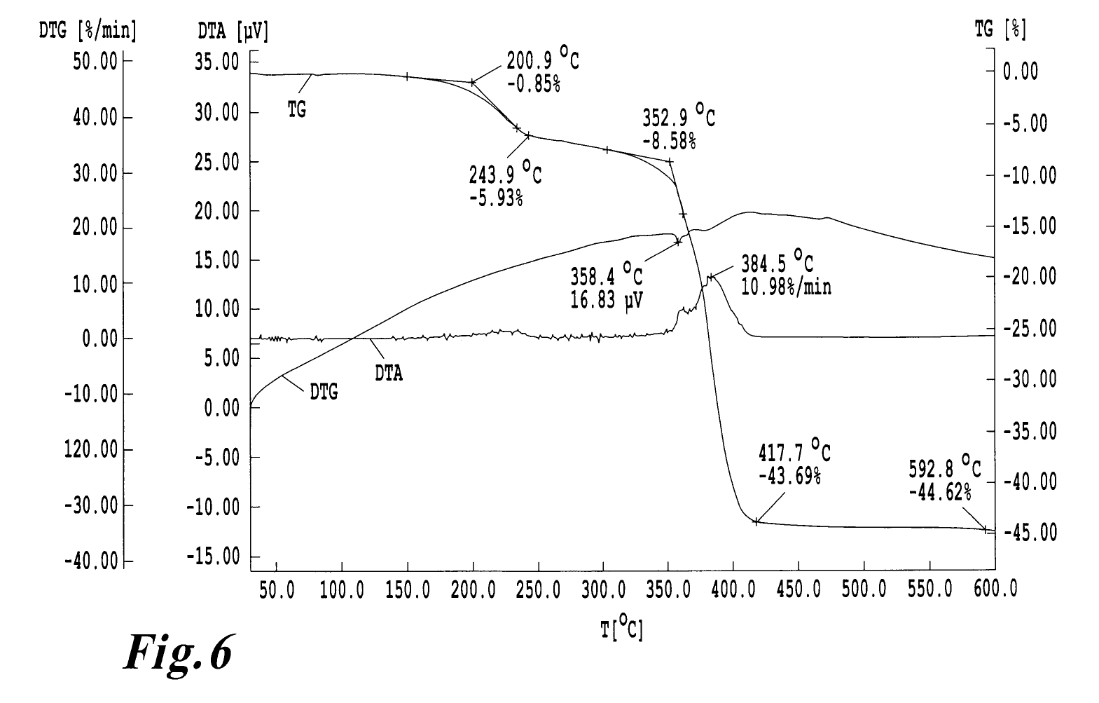

A sample of N,N′-bis(4,6-dipropylnon-5-yl)quaterrylene-3,4:13,14-tetracarboximide (6.970 mg) was weighed and analyzed thermogravimetrically under a nitrogen atmosphere. The temperature was increased with a gradient of 10° C./min from 30° C. to 600° C. The results as a function of temperature are reproduced in FIG. 6. Up to a temperature of 243.9° C., there was a weight loss of 5.93%, which can be attributed to the loss of solvent. Up to a temperature of 417.7° C., there was a weight loss of 43.69%. This corresponds approximately to the weight loss of the two 4,6-dipropylnonyl groups and the solvent loss, based on the total weight of the compound used. No further decomposition was observed within the analysis range.

The temperature of the DTG peak maximum, which corresponds to the temperature of the maximum reaction conversion, was observed at 384.5° C. with a maximum of 10.98%/min. The decomposition temperature in the case of compounds of the formula (II) which bear an alkyl group with a double branch on the imide nitrogen atom was thus lower than in the case of compounds of the formula (II) which bear an alkyl group with a single branch on the imide nitrogen atom.

### Example I.5

### Decomposition of N,N′-bis(1-ethylbenzyl)perylene-3,4:9,10-tetracarboximide

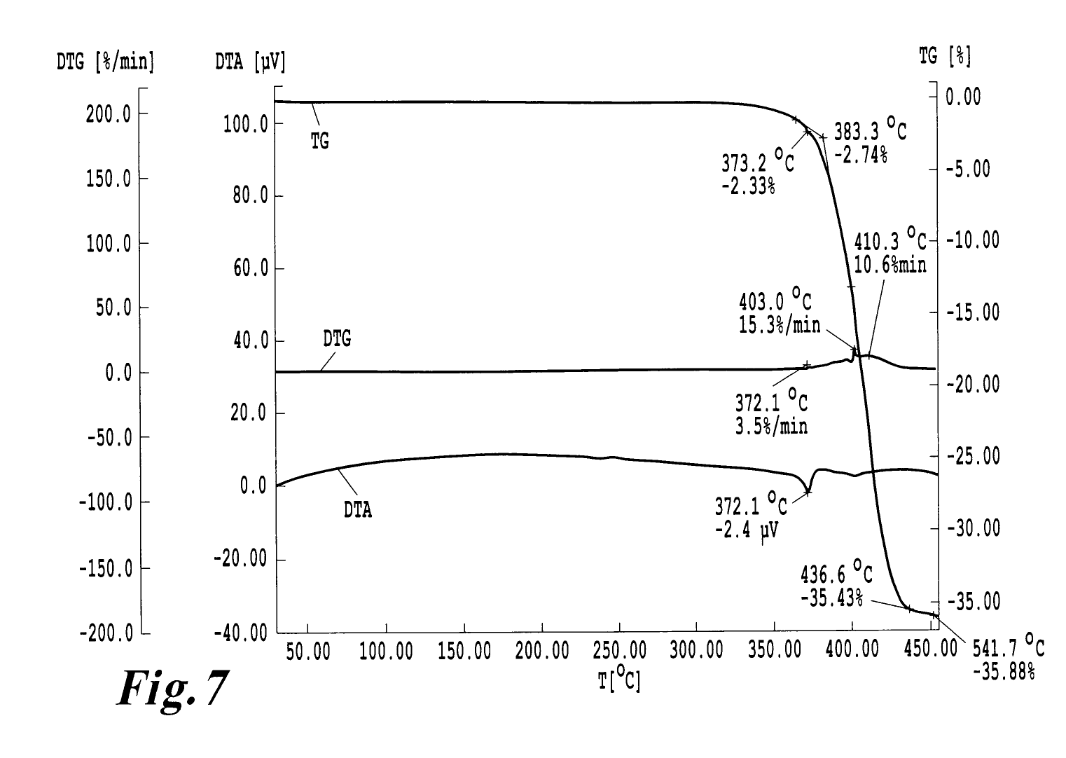

A sample of N,N′-bis(1-ethylbenzyl)perylene-3,4:9,10-tetracarboximide (8.025 mg) was weighed and analyzed thermogravimetrically under a nitrogen atmosphere. The temperature was increased with a gradient of 10° C./min from 30° C. to 450° C. The results as a function of temperature are reproduced in FIG. 7. A defined decrease in the weight of the sample was observed within a temperature range of from 370 to 450° C. with maxima of 15.3%/min at a temperature of 403° C. and 10.6%/min at 410.3° C. The weight of the sample decreased by 36%. This corresponds approximately to the proportion by weight of the two 1-ethylbenzyl groups, based on the total weight of the compound used. No further decomposition is observed within the analysis range.

The decomposition temperature in the case of compounds of the formula (II) which bear a 1-alkylbenzyl group on the imide nitrogen was thus lower than in the case of compounds of the formula (II) which bear an alkyl group with a single branch on the imide nitrogen atom, but was higher than in the case of compounds of the formula (II) which bear an alkyl group with a double branch on the imide nitrogen atom. The stabilization by a phenyl group leads to a lowering of the decomposition temperature.

### II) Preparation of Compounds of the Formula (II)

### Example II.1

### Preparation of 1,6,9,14-tetracyano-N,N′-di(1-heptyloctyl)terrylene-3,4:11,12-tetracarboximide

### a) Provision of N,N′-di(1-heptyloctyl)terrylene-3,4:11,12-tetracarboximide by single-stage base-induced dimerization

A mixture of diethylene glycol diethyl ether (7 ml), sodium tert-butoxide (2.79 g, 29 mmol) and diazabicycloundecene (DBU, 13.7 g, 90 mmol) was admixed at 60° C. with N-(1-heptyloctyl)perylene-3,4-dicarboxylic monoimide (0.77 g, 1.45 mmol) and N-(1-heptyloctyl)napthalene-1,8-dicarboxylic monoimide (2.36 g, 5.8 mmol). The reaction mixture was stirred at 130° C. for 6 hours. After cooling to room temperature, the reaction mixture was diluted with ethyl acetate and washed repeatedly with dilute hydrochloric acid, dried over magnesium sulfate and freed of the solvent under reduced pressure. N,N′-Di(1-heptyloctyl)terrylene-3,4:11,12-tetracarboximide is isolated from the residue by column chromatography (SiO₂, toluene/petroleum ether, gradient). 0.13 g of a blue solid is obtained (10% yield).

### b) Provision of N,N′-Di(1-heptyloctyl)-1,6,9,14-tetrabromoterrylene-3,4:11,12-tetracarboximide

N,N′-Di(1-heptyloctyl)terrylene-3,4:11,12-tetracarboximide (from step a, 0.13 g, 0.13 mmol) was taken up in a mixture of chlorobenzene (15 ml) and water (5 ml). The reaction mixture thus obtained was admixed with a few drops of bromine and a spatula-tip of iodine and stirred at a temperature of 90° C. for 7 hours. After cooling to room temperature, the reaction mixture was diluted with dichloromethane and admixed with an aqueous solution of sodium sulfite. After the phases had been separated, the organic phase was dried and freed of the solvent under reduced pressure. N,N′-Di(1-heptyloctyl)-1,6,9,14-tetrabromoterrylene-3,4:11,12-tetracarboximide was isolated from the residue by column chromatography (SiO₂, toluene/petroleum ether, gradient). 90 mg of a blue solid were obtained (52% yield). Rf (toluene)=0.71.

### c) Preparation of N,N′-di(1-heptyloctyl)-1,6,9,14-tetracyanoterrylene-3,4:11,12-tetracarboximide

A mixture of N,N′-di(1-heptyloctyl)-1,6,9,14-tetrabromoterrylene-3,4:11,12-tetracarboximide (from step b, 50 mg), zinc cyanide (169 mg, 1.44 mmol), 1,1′-diphenylphosphinoferrocene (10 mg, 0.018 mmol) and tris(dibenzylidene-acetone)dipalladium (16.5 mg, 0.018 mmol) in toluene (10 ml) was stirred at 80° C. for 45 hours and at 100° C. for a further 22 hours. Subsequently, the reaction mixture was admixed with dichloromethane and water. After the phases had been separated, the organic phase was dried and freed of the solvent under reduced pressure. N,N′-Di(1-heptyloctyl)-1,6,9,14-tetracyanoterrylene-3,4:11,12-tetracarboximide is isolated from the residue by column chromatography. Rf (CH₂Cl₂)=0.34.

### Example 11.2

### Preparation of N,N′-bis(1-heptyloctyl)-1,6,7,12-tetrachloroperylene-3,4:9,10-tetracarboximide

A mixture of imidazole (50 g), tetrachloroperylene-3,4:9,10-tetracarboxylic dianhydride (658 mg, 1.25 mmol) and 1-heptyloctylamine (0.57 g, 2.5 mmol) was heated to 90° C. for 2 hours. Subsequently, the reaction mixture was admixed again with 1-heptyloctyl-amine (0.57 g, 2.5 mmol) and stirred at 90° C. for a further 2 hours. After cooling to room temperature, the reaction mixture was poured onto water and extracted with dichloromethane. The resulting organic phases were combined, dried over magnesium sulfate and freed of the solvent under reduced pressure. N,N′-Bis(1-heptyloctyl)-1,6,7,12-tetrachloroperylene-3,4:9,10-tetracarboximide was isolated from the residue by column chromatography (SiO₂, toluene) as an orange solid in an amount of 0.18 g (15% yield). The solubility of the resulting compound in toluene is >15%. Rf (toluene)=1.

### Example II.3

### Preparation of N,N′-bis(4,6-dipropylnon-5-yl)quaterrylene-3,4:13,14-tetracarboximide

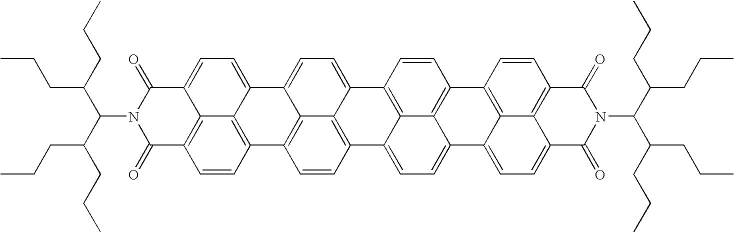

### a) Provision of N-(4,6-dipropylnon-5-yl)perylene-3,4-dicarboxylic monoimide

A mixture of 24 ml of quinoline, 3.6 g (16.5 mmol) of zinc acetate dihydrate, 0.94 g (5.5 mmol) 4,6-dipropylnon-5-ylamine (prepared according to DE 10 2004 024909) and 2.05 g (5.5 mmol) of perylene-3,4-dicarboxylic monoanhydride (prepared according to Liebigs Annalen 1995, 7, 1229-1244) was heated to 160° C. for 2 hours. A further 0.94 g (5.5 mmol) of 4,6-dipropylnon-5-ylamine was added and the mixture was heated at 180° C. and for a further 2 hours. After the reaction mixture had been cooled, it was poured onto 150 ml of conc. Hydrochloric acid, and the precipitate was filtered, washed with hot water and dried. 2.62 g of an orange crude product were obtained, which was purified by column chromatography with toluene. 0.9 g (34%) of the orange title compound was obtained.

### b) Preparation of N,N′-bis(4,6-dipropylnon-5-yl)quaterrylene-3,4:13,14-tetracarboximide

A mixture of 0.70 g (6.2 mmol) of potassium tert-butoxide, 1.09 g (7.2 mmol) of diazabicyclo[5.4.0]undec-7-ene, 17.0 g (17 mmol) of ethanolamine and 0.43 g (0.81 mmol) of the perylenemonoimide compound obtained in example II.3.a) was heated to 140° C. for 11 hours. A further 0.7 g (6.2 mmol) of potassium tert-butoxide, 1.09 g (7.2 mmol) of diazabicyclo[5.4.0]undec-7-ene and 17.0 g (17 mmol) of ethanolamine were added, and the mixture was stirred at 140° C. for a further 29 hours. The reaction mixture was allowed to cool and poured onto 60 ml of concentrated hydrochloric acid solution. The precipitate was filtered off, washed with water and dried under reduced pressure. 0.5 g of a green crude product were obtained, which was purified by column chromatography on silica gel. First, ethyl acetate, methanol and ethanol were used to wash impurities from the column, before the product was eluted with dichloromethane. 100 mg (23% yield) of the title compound were obtained.

### Example II.4

### Preparation of N,N′-bis(1-ethylbenzyl)perylene-3,4:9,10-tetracarboximide

A mixture of 25 ml of quinoline, 1.0 g of zinc acetate dihydrate, 2.08 g (5.1 mmol) of perylene-3,4:9,10-tetracarboxylic dianhydride and 2.14 g (15.4 mmol) of 1-ethylbenzylamine was heated to 200° C. for 72 hours. Subsequently, the reaction mixture was cooled and precipitated in conc. hydrochloric acid solution, and the precipitate was filtered off and dried under reduced pressure. 3.2 g (88%) of a red residue were obtained. The residue was purified repeatedly by column chromatography on silica gel (2:3 toluene/dichloromethane) and subsequent crystallization (4:1 toluene/dichloromethane).

III. Performance Properties when Used in OFETs:

### Production of the OFET

The substrates used were n-doped silicon wafers (2.5×2.5 cm, conductivity <0.004 Ω⁻¹cm) with a thermally deposited oxide layer (300 nm) as a dielectric (area-based capacitance Cᵢ=10 nF/cm²). The coated substrates were cleaned by rinsing with toluene, acetone and isopropanol and dried under a nitrogen stream. Subsequently, the wafers were immersed into a solution composed of 3 ml of phenyltriethyloxysilane and 100 ml of toluene for 12 hours. Thereafter, the wafers were washed again with toluene, acetone and isopropanol, before they were dried in a nitrogen stream.

### Application by Spin-Coating

The compounds of the formula (II) were, unless mentioned otherwise, dissolved in chloroform (5 mg/ml) and spin-coated onto the substrates produced as above at 1000 rpm within 30 seconds.

### Application by Shearing

The compounds of the formula (II) were, unless mentioned otherwise, dissolved in chlorobenzene (5 mg/ml) and applied dropwise to the silicon dioxide/silicon wafer. A further silicon dioxide/silicon wafer which had been hydrophobized by the above-specified method using octyltriethyoxysilane was coated using a syringe pump, so as to form a film. This was done by pushing a plunger in a syringe forward very slowly with a defined speed with an electric motor. The shear rate is specified in table 5.

### Drying and Controlled Thermolysis

The films produced by spin-coating or shearing were dried at 70° C. under reduced pressure (approx. 100 mbar) for 12 hours. Subsequently, the dried films were heated at the temperature specified in a nitrogen-filled glovebox.

### Completion of the OFET

Source and drain electrodes made of gold, of channel length 50 μm and length/width ratio of about 20, were applied to the films by means of a shadowmask.

The results of the testing of the transistor properties are reproduced in tables 1 to 4 and table 6. The measurements were effected under a nitrogen atmosphere.

### Example III.1

### OFET Comprising the Compound NH,NH′-quaterrylene-3,4:9,10-tetracarboximide

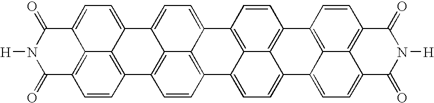

prepared by thermolysis of N,N′-bis((1-hexylheptyl)quaterrylene-3,4:13,14-tetracarboximide

TABLE 1

| | | 400° C., 1 h | 380° C., 30 min |
|---|---|---|---|
| spin- | mobility μₘₐₓ (cm²/Vs) | 7.4 × 10⁻³ | 3.5 × 10⁻³ |
| coating | Iₒₙ/I_{off} | 2.5 × 10⁴ | 1.4 × 10³ |
| | Vₜ (V) | 11.9 | 0.4 |
| shearing | mobility μₘₐₓ (cm²/Vs) | 8.8 × 10⁻² | 4.4 × 10⁻³ |
| | Iₒₙ/I_{off} | 2.5 × 10³ | 7.0 × 10² |
| | Vₜ (V) | −1.8 | −12.6 |

### Example III.2

### OFET comprising NH,N′H-1,6,7,12-tetrachloroperylene-3,4:9,10-tetracarboximide

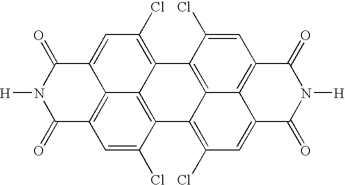

prepared by thermolysis of N,N′-bis(1-heptyloctyl)-1,6,7,12-tetrachloroperylene-3,4:9,10-tetracarboximide from example II.2

TABLE 2

| | | 380° C., 30 min |
|---|---|---|
| shearing | mobility μ (cm²/Vs) | 1.78 × 10⁻³ |
| | Iₒₙ/I_{off} | 2.1 × 10³ |
| | Vₜ (V) | −2.7 |

### Example III.3

### OFET comprising NH,N′H-terrylene-3,4:11,12-tetracarboximide

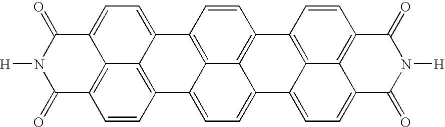

prepared by thermolysis of N,N′-bis(1-heptyloctyl)-terrylene-3,4:11,12-tetracarboximide

TABLE 3

| | | 380° C., 30 min |
|---|---|---|
| shearing | mobility μ (cm²/Vs) | 8.0 × 10⁻⁶ |
| | Iₒₙ/I_{off} | 1.3 × 10² |
| | Vₜ (V) | 49.9 |

### Example III.4

### OFET comprising NH,N′H-terrylene-3,4:11,12-tetracarboximide

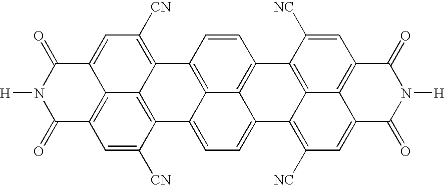

prepared by thermolysis of 1,6,9,14-tetracyano-N,N′-di(1-heptyloctyl)terrylene-3,4:11,12-tetracarboximide from example II.1

TABLE 4

| | | 380° C., 30 min |
|---|---|---|
| spin-coating | mobility μ (cm²/Vs) | 9.7 × 10⁻⁶ |
| | Iₒₙ/I_{off} | 1.9 × 10² |
| | Vₜ (V) | −3.0 |
| shearing | mobility μ (cm²/Vs) | 4.3 × 10⁻⁶ |
| | Iₒₙ/I_{off} | 2.5 × 10² |
| | Vₜ (V) | 14.0 |

### Example III.5

### OFET Comprising

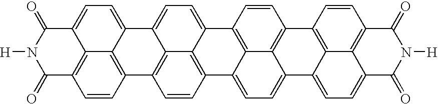

prepared by thermolysis of N,N′-bis(4,6-dipropylnon-5-yl)quaterrylene-3,4:13,14-tetracarboximide from example III.2.b).

The films were obtained by spin-coating from a 5 mg/ml solution at 1000 rpm of the chlorinated solvent specified (table 5). Films were likewise obtained by shearing at the shear rates specified (table 5). In the case of shearing, chlorobenzene was used as the solvent.

The thermolysis in experiments III.5.B, III.5.C, III.5.D, III.5.E, III.5F, III.5.G, III.5.H, III.5.J, III.5.K and III.5.L was carried out under the action of pressure (table 5). First, the film obtained by shearing or spin-coating was dried at 80° C. in a vacuum drying cabinet (100 mbar). Thereafter, during the thermolysis, a glass plate was placed onto the film, which pressed onto the film with a weight with the pressure specified.

In all examples, the thermolysis was carried out at 370° C. over a period of 1 hour.

TABLE 5

| | | Concen- | | Pres- | | |
|---|---|---|---|---|---|---|
| | | tration | | sure | Shear rate | Speed of |
| Ex. | Method | [mg/ml] | Solvent | [kPa] | [mm/sec] | rotation [rpm] |
| III.5.A | shearing | 5 | chlorobenzene | 0.0 | 0.86 | — |
| III.5.B | shearing | 5 | chlorobenzene | 5.2 | 0.43 | — |
| III.5.C | shearing | 5 | chlorobenzene | 5.2 | 1.29 | — |
| III.5.D | shearing | 5 | chlorobenzene | 5.2 | 0.86 | — |
| III.5.E | shearing | 5 | chlorobenzene | 20.9 | 0.86 | — |
| III.5.F | shearing | 10 | chlorobenzene | 59.1 | 0.86 | — |
| III.5.G | spin-coating | 5 | chlorobenzene | 5.2 | — | 1000 |
| III.5.H | spin-coating | 5 | chloroform | 5.2 | — | 1000 |
| III.5.I | spin-coating | 10 | chlorobenzene | 0.0 | — | 1000 |
| III.5.J | spin-coating | 10 | chlorobenzene | 10.3 | — | 1000 |
| III.5.K | spin-coating | 10 | chlorobenzene | 20.5 | — | 1000 |
| III.5.L | spin-coating | 10 | chlorobenzene | 59.1 | — | 1000 |

The results of the testing of the transistor properties are reproduced in table 6.

TABLE 6

| | Mobility | | |
|---|---|---|---|
| Ex. | [cm²/Vs] | On/off | V_{threshold} (V) |
| III.5.A | 9.5 × 10⁻⁴ | 3.0 × 10⁴ | 15 |
| III.5.B | 1.5 × 10⁻² | 1.4 × 10⁴ | 17 |
| III.5.C | 1.8 × 10⁻³ | 2.2 × 10⁴ | 24 |
| III.5.D | 1.6 × 10⁻² | 2.9 × 10³ | −1 |
| III.5.E | 6.0 × 10⁻² | 5.6 × 10⁴ | −1 |
| III.5.F | 3.9 × 10⁻² | 4.9 × 10³ | 2 |
| III.5.G | 5.1 × 10⁻⁴ | 3.6 × 10⁴ | 18 |
| III.5.H | 3.3 × 10⁻⁸ | 1.5 × 10² | 22 |
| III.5.I | 5.1 × 10⁻⁴ | 3.6 × 10⁴ | n.d. |
| III.5.J | 2.8 × 10⁻³ | 1.3 × 10⁴ | n.d. |
| III.5.K | 4.2 v10⁻³ | 1.1 × 10⁴ | n.d. |
| III.5.L | 6.8 × 10⁻⁴ | 4.7 × 10³ | n.d. |

n.d. not determined

It can be seen that the thermolysis has a positive effect on the field-effect mobilities.

### III.6 Decomposition of N,N′-bis(1-heptyloctyl)quaterrylene-3,4:13,14-tetracarboximide on FTO glass

One measurement was carried out using the technique of the desorption spectrum (TDS), in order to examine the compound of the formula (I) prepared in step iii) of the process according to the invention.

FTO glass (fluorine-doped tin oxide) was used as the substrate of a solar cell. N,N′-Bis(1-heptyloctyl)quaterrylene-3,4:13,14-tetracarboximide was applied to FTO glass as described above by shearing (1 mm/sec and heated to 400° C. under nitrogen for one hour. After mixing with dihydroxybenzyl alcohol, a matrix-assisted laser desorption spectrum of the organic layer was recorded. The cationic spectrum shows merely the NH,N′H-quaterrylenediimide compound, whereas the anionic spectrum, as well as this main compound, exhibits a trace of NH,N′-1-(heptyloctyl)quaterrylene-3,4:13,14-tetracarboximide, i.e. of the singly decomposed compound.

This experiment shows that compounds of the formula (II) can be decomposed in sufficient purity on a material suitable for solar cells.

1. A process for producing a substrate coated at least partly with a compound of the formula (I) 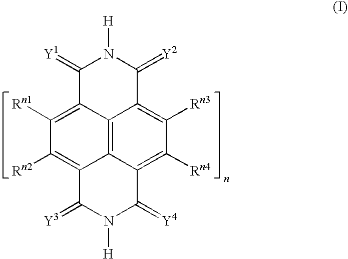
in which
n is an integer from 1 to 8
Y¹, Y², Y³ and Y⁴ are each independently O or S and
Rⁿ¹, Rⁿ², Rⁿ³ and Rⁿ⁴ are each independently hydrogen, F, Cl, Br, CN, alkoxy, alkylthio, alkylamino, dialkylamino, aryloxy, arylthio, hetaryloxy or hetarylthio, where
two of the Rⁿ¹ and Rⁿ² radicals and/or Rⁿ³ and Rⁿ⁴ radicals in each case may together also be part of an aromatic ring system fused to one or two adjacent naphthalene units of the rylene skeleton;
in which
i) at least one compound of the formula (II) is provided 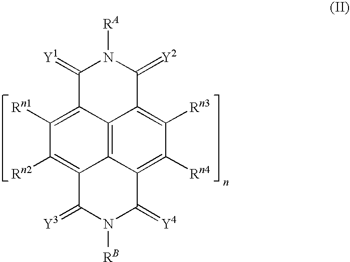
in which
n, Y¹, Y², Y³, Y⁴, Rⁿ¹, Rⁿ², Rⁿ³ and Rⁿ⁴ are each as defined for the compound of the formula (I) and
R^{A} and R^{B} are each independently a group of the formula (III) 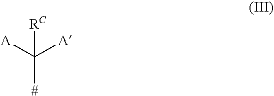
in which
# in each case represents the bond to the nitrogen atom,
A and A′ are each independently in each case optionally substituted C₁-C₂₅-alkyl, C₂-C₂₅-alkenyl, C₂-C₂₅-alkynyl, aryl or hetaryl, where C₁-C₂₅-alkyl, C₂-C₂₅-alkenyl and C₂-C₂₅-alkynyl may each be interrupted once or more than once by O, S, NR^{a}, —C(═O)—, —C(═O)O—, —C(═O)N(R^{a})—, —S(═O)₂O— or —S(═O)₂N(R^{a})—, in which
R^{a} is selected from in each case optionally substituted C₁-C₁₂-alkyl, aryl and hetaryl, and
R^{C} is hydrogen or in each case optionally substituted C₁-C₁₂-alkyl, C₂-C₁₂-alkenyl, C₂-C₁₂-alkynyl, aryl or hetaryl, where C₁-C₂₅-alkyl, C₂-C₂₅-alkenyl and C₂-C₂₅-alkynyl may each be interrupted once or more than once by O, S, NR^{a}, —C(═O)—, —C(═O)O—, —C(═O)N(R^{a})—, —S(═O)₂O— or —S(═O)₂N(R^{a})—,
ii) the substrate is treated with a solution of the compounds of the formula (II) provided and
iii) the treated substrate is heated to a temperature at which at least some of the compounds of the formula (II) are converted to compounds of the formula (I).

2. The process according to claim 1, wherein at least one of the A and A′ radicals in the groups of the formula (III) is in each case optionally substituted C₁-C₂₅-alkyl, C₃-C₂₅-alkenyl or C₃-C₂₅-alkynyl with at least one hydrogen atom in the beta position to the nitrogen atom of the rylene skeleton, where C₁-C₂₅-alkyl, C₃-C₂₅-alkenyl and C₃-C₂₅-alkynyl may each be interrupted once or more than once by O, S, NR^{a}, —C(═O)—, —C(═O)O—, —C(═O)N(R^{a})—, —S(═O)₂O— or —S(═O)₂N(R^{a})—, and in which R^{a} is as defined in claim 1.

3. The process according to claim 1 or 2, in which the Y¹, Y², Y³ and Y⁴ radicals in the compounds of the formula (I) and (II) are each O.

4. The process according to any one of the preceding claims, in which the Rⁿ¹, Rⁿ², Rⁿ³ and Rⁿ⁴ radicals in the compounds of the formula (I) and (II) are each independently hydrogen, F, Cl, Br, CN, aryloxy or arylthio.

5. The process according to any one of claims 1 to 4, in which, in the group of the formula (III), at least one of the A or A′ radicals is a —CH(R^{D})(R^{E}) group in which R^{D} and R^{E} are each independently C₁-C₁₂-alkyl or in each case optionally substituted aryl or hetaryl, where C₁-C₁₂-alkyl may be interrupted once or more than once by O or S.

6. The process according to claim 5, in which R^{c} is hydrogen or in each case optionally substituted C₁-C₁₂-alkyl, aryl or hetaryl, where C₁-C₁₂-alkyl may be interrupted once or more than once by O or S.

7. The process according to any one of claims 1 to 4, in which, in the group of the formula (III), one of the A and A′ radicals is a —CH(R^{D})(R^{E}) group in which R^{D} and R^{E} are each independently C₁-C₁₂-alkyl or in each case optionally substituted aryl or hetaryl, and where C₁-C₁₂-alkyl may be interrupted once or more than once by O or S and the other of the A and A′ radicals is in each case optionally substituted aryl or hetaryl.

8. The process according to any one of the preceding claims, in which, in the compounds of the formula (II),
A and A′ are each independently a —CH(R^{D})(R^{E}) group in which R^{D} and R^{E} are each independently hydrogen or C₁-C₁₂-alkyl, and
R^{C} is hydrogen or C₁-C₁₂-alkyl.

9. The process according to any one of the preceding claims, in which, in step ii), the substrate is treated with introduction of shearing energy.

10. The process according to any one of the preceding claims, in which the treated substrate is heated in step iii) to a temperature in the range from 200 to 600° C.

11. The process according to any one of the preceding claims, in which the treated substrate is heated in step iii) under the action of pressure.

12. The process according to any one of the preceding claims, in which the substrate is additionally treated with a thermally stable, electron-rich compound which is suitable for doping the layer of the compounds of the formula (I), or with a compound which is converted under the conditions of the heating in step iii) to such an electron-rich compound.

13. A coated substrate obtainable by a process as defined in any one of claims 1 to 12.

14. The substrate according to claim 13, comprising at least one compound of the formula (I) as emitter materials, charge transport materials or exciton transport materials.

15. The substrate according to claim 14, comprising at least one organic field-effect transistor, comprising a gate structure, a source electrode and a drain electrode.

16. A semiconductor component comprising at least one substrate as defined in claim 13.

17. An organic solar cell, especially in the form of an excitonic solar cell, comprising at least one substrate as defined in claim 13.

18. An organic light-emitting diode comprising at least one substrate as defined in claim 13.

19. A process for preparing compounds of the formula (I) as defined in claim 1, in which
A) a compound of the formula (II) as defined in any one of claims 1 to 8 is provided,
B) the compound of the formula (II) is heated to a temperature at which at least some of the compound of the formula (II) is converted to a compound of the formula (I).

20. A compound of the formula (I)′ 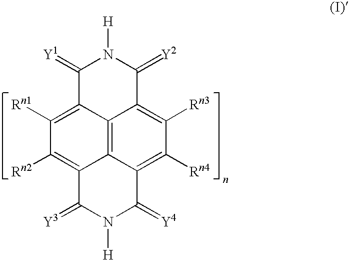
in which
n is 4,
Y¹, Y², Y³ and Y⁴ are each independently O or S and
Rⁿ¹, Rⁿ², Rⁿ³ and Rⁿ⁴ are each independently hydrogen or cyano, where two of the Rⁿ¹ and Rⁿ² radicals and/or Rⁿ³ and Rⁿ⁴ radicals in each case may together also be part of an aromatic ring system fused to one or two adjacent naphthalene units of the rylene skeleton,
where at least one of the Rⁿ¹, Rⁿ², Rⁿ³ and Rⁿ⁴ radicals is CN.

21. A compound of the formula (II)′ 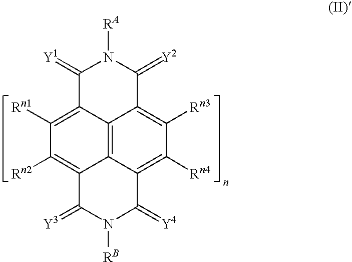
in which
n is 3 or 4,
Y¹, Y², Y³ and Y⁴ are each independently O or S,
Rⁿ¹, Rⁿ², Rⁿ³ and Rⁿ⁴ are each independently hydrogen or cyano, where two of the Rⁿ¹ and Rⁿ² radicals and/or Rⁿ³ and Rⁿ⁴ radicals in each case may together also be part of an aromatic ring system fused to one or two adjacent naphthalene units of the rylene skeleton,
where at least one of the Rⁿ¹, Rⁿ², Rⁿ³ and Rⁿ⁴ radicals is CN,
R^{A} and R^{B} are each independently a group of the formula (III) 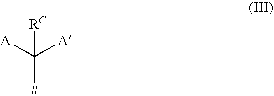
in which
# in each case represents the bond to the nitrogen atom,
A and A′ are each independently in each case optionally substituted C₀-C₂₅-alkyl, C₂-C₂₅-alkenyl, C₂-C₂₅-alkynyl, aryl or hetaryl, where C₁-C₂₅-alkyl, C₂-C₂₅-alkenyl and C₂-C₂₅-alkynyl may each be interrupted once or more than once by O, S, NR^{a}, —C(═O)—, —C(═O)O—, —C(═O)N(R^{a})—, —S(═O)₂O— or —S(═O)₂N(R^{a})—, in which
R^{a} is selected from in each case optionally substituted C₁-C₁₂-alkyl, aryl and hetaryl, and
R^{C} is hydrogen or in each case optionally substituted C₁-C₁₂-alkyl, C₂-C₁₂-alkenyl, C₂-C₁₂-alkynyl, aryl or hetaryl, where C₁-C₂₅-alkyl, C₂-C₂₅-alkenyl and C₂-C₂₅-alkynyl may each be interrupted once or more than once by O, S, NR^{a}, —C(═O)—, —C(═O)O—, —C(═O)N(R^{a})—, —S(═O)₂O— or —S(═O)₂N(R^{a})—.

22. A compound of the formula (II)′ according to claim 21, wherein at least one of the A and A′ radicals in the groups of the formula (III) is in each case optionally substituted C₁-C₂₅-alkyl, C₃-C₂₅-alkenyl or C₃-C₂₅-alkynyl with at least one hydrogen atom in the beta position to the nitrogen atom of the rylene skeleton, where C₁-C₂₅-alkyl, C₃-C₂₅-alkenyl and C₃-C₂₅-alkynyl may each be interrupted once or more than once by O, S, NR^{a}, —C(═O)—, —C(═O)O—, —C(═O)N(R^{a})—, —S(═O)₂O— or —S(═O)₂N(R^{a})—, and in which R^{a} is as defined in claim 21.

23. A compound of the formula (II)′ according to claim 21 or 22, in which A and A′ in the groups of the formula (III) are each C₄-C₂₅-alkyl.

24. A compound of the formula (I)′ according to claim 20 or a compound of the formula (II)′ according to any one of claims 21 to 23, in which from 1 to (2n−2) of the Rⁿ¹, Rⁿ², Rⁿ³ and Rⁿ⁴ radicals are CN.

25. A process for preparing compounds of the formula (II)′ according to any one of claims 21 to 23, in which a compound of the formula (II)′ in which at least one of the Rⁿ¹, Rⁿ², Rⁿ³ and Rⁿ⁴ radicals is bromine or chlorine is subjected to a reaction with a monovalent or divalent metal cyanide in an aromatic hydrocarbon as the solvent to obtain compounds of the formula (II)′ in which at least one of the Rⁿ¹, Rⁿ², Rⁿ³ and Rⁿ⁴ radicals is cyano.

26. The use of a solution of compounds of the formula (II)′ according to any one of claims 21 to 24 for treatment of substrates to coat these substrates over at least part of their surface area with compounds of the formula (II)′.

27. A compound of the formula (II)″ 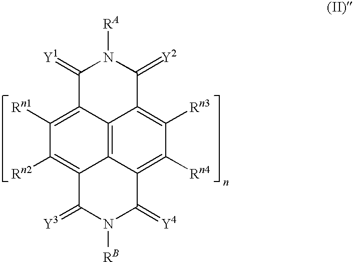
in which
n is an integer from 1 to 8
Y¹, Y², Y³ and Y⁴ are each independently O or S and
Rⁿ¹, Rⁿ², Rⁿ³ and Rⁿ⁴ are each independently hydrogen, F, Cl, Br, CN, alkoxy, alkylthio, alkylamino, dialkylamino, aryloxy, arylthio, hetaryloxy or hetarylthio, where two of the Rⁿ¹ and Rⁿ² radicals and/or Rⁿ³ and Rⁿ⁴ radicals in each case may together also be part of an aromatic ring system fused to one or two adjacent naphthalene units of the rylene skeleton, and
R^{A} and R^{B} are each independently a group of the formula (III)′ 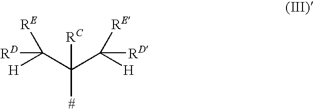
in which
# in each case represents the bond to the nitrogen atom,
R^{C} is hydrogen or C₁-C₁₂-alkyl and
R^{D}, R^{D′}, R^{E} and R^{E′} are each independently C₁-C₁₂-alkyl,
excluding N,N′-bis(1-isopropyl-2-methylpropyl)perylene-3,4:9,10-tetra-carboximide, N,N′-bis[2-ethyl-1-(1-ethylpropyl)butyl]perylene-3,4:9,10-tetracarboximide and N,N′-bis[2-propyl-1-(1-propylbutyl)pentyl]perylene-3,4:9,10-tetracarboximide.

28. The use of a solution of compounds of the formula (II)″ according to claim 27 for treatment of substrates to coat these substrates over at least part of their surface area with compounds of the formula (II)″.

29. A compound of the formula (II)′″ 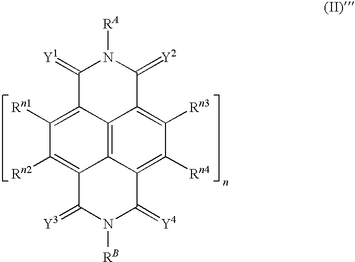
in which
n is an integer from 5 to 8
Y¹, Y², Y³ and Y⁴ are each independently O or S and
Rⁿ¹, Rⁿ², Rⁿ³ and Rⁿ⁴ are each independently hydrogen, F, Cl, Br, CN, alkoxy, alkylthio, alkylamino, dialkylamino, aryloxy, arylthio, hetaryloxy or hetarylthio, where two of the Rⁿ¹ and Rⁿ² radicals and/or Rⁿ³ and Rⁿ⁴ radicals in each case may together also be part of an aromatic ring system fused to one or two adjacent naphthalene units of the rylene skeleton, and
R^{A} and R^{B} are each independently a group of the formula (III) 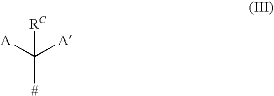
in which
# in each case represents the bond to the nitrogen atom,
A and A′ are each independently in each case optionally substituted C₁-C₂₅-alkyl, C₂-C₂₅-alkenyl, C₂-C₂₅-alkynyl, aryl or hetaryl, where C₁-C₂₅-alkyl, C₂-C₂₅-alkenyl and C₂-C₂₅-alkynyl may each be interrupted once or more than once by O, S, NR^{a}, —C(═O)—, —C(═O)O—, —C(═O)N(R^{a})—, —S(═O)₂O— or —S(═O)₂N(R^{a})—, in which
R^{a} is selected from in each case optionally substituted C₁-C₁₂-alkyl, aryl and hetaryl, and
R^{C} is hydrogen or in each case optionally substituted C₁-C₁₂-alkyl, C₂-C₁₂-alkenyl, C₂-C₁₂-alkynyl, aryl or hetaryl, where C₁-C₂₅-alkyl, C₂-C₂₅-alkenyl and C₂-C₂₅-alkynyl may each be interrupted once or more than once by O, S, NR^{a}, —C(═O)—, —C(═O)O—, —C(═O)N(R^{a})—, —S(═O)₂O— or —S(═O)₂N(R^{a})—,
excluding N,N′-bis(1-heptyloctyl)pentarylene-3,4:17,18-tetracarboximide.

30. A compound of the formula (II)′″ according to claim 29, in which at least one of the A and A′ radicals in the groups of the formula (III) is in each case optionally substituted C₁-C₂₅-alkyl, C₃-C₂₅-alkenyl or C₃-C₂₅-alkynyl with at least one hydrogen atom in the beta position to the nitrogen atom of the rylene skeleton, where C₁-C₂₅-alkyl, C₃-C₂₅-alkenyl and C₃-C₂₅-alkynyl may each be interrupted once or more than once by O, S, NR^{a}, —C(═O)—, —C(═O)O—, —C(═O)N(R^{a})—, —S(═O)₂O— or —S(═O)₂N(R^{a})—, and in which R^{a} is as defined in claim 29.

31. The use of a solution of compounds of the formula (II)′″ according to either of claims 29 and 30 for treatment of substrates to coat these substrates over at least part of their surface area with compounds of the formula (II)′″.
